SEQUENCE LISTING (13 sequences)

SEQ ID NO: 1 - DNA, 248 bp, Unknown
  Region 1 of the consensus sequence of NBL2
aygtggtttgggttaggtatagacgtcccccacaccctccgggtccccggtgttgtttgattttccttggcattgacggaaaggtcacccgtttccgccttccaccggcatatgcctggaccccacccttcgtttcgccgtcgccccgtttgcctccggtgtcacacgttaacaccaactgctgtgggattggccagtgccacgcgtggtcacatggtctccccctagcgatgcgttcgagcatcgct

SEQ ID NO: 2 - DNA, 1410 bp, Unknown
  NBL2 Consensus sequence
gcggccgccgcgcggcggtggcgatatttaaaggggacgcagcctatctgtcaggagtggagcgtgagtcggctcagccaatgcgcatgcgctaggcgccagcggtttctcccgtcacagtgattcccacggttgtcttagacactagtccccgaggcttggcagagcaggagccctccgtggcagtgcttgggtatccaggctctgaggctccggcctcacctttccacggggtcgaagggaacctctcctgatgacagcagtcgcaaagggcggaccatgatgatgataccccaggcggagacgggggaagcagcacgggatcccagcctcaggcctgcacggacggtgttggttggggtgagtctcaccaaaagtcgtgccggcgtccgtgatctcgaggacgggtcggcctgcgtgcccctcggctgctctctcacccgagggtcattctcctccagagcagaaccccggatcctcaggggttgcctggtgttgtgtgtttcaatgcctctgctgtatgactctgcgtgtgtgtgcgtgtgtgtctgtgtgtgtgtctcccattctctcttctctctctgtctctcactctctgtgtgtttctttcccactctctgtgggtttgtaccagccgaagttgcgtcagggctaccaggtcggtggaggattgggggtgttgcgaaattggcagaaacctctttgctcctctgggaggcatttgaaaacgtggcttgggtcaggcacaggaggcccccctaccccacggttcccaggtgttgtttgtttttccttgacgttgacgaaaatgtcacctgtttcccccttccaccggcatatccctggaccccacccttcgttttgccgtcgccccgtatgcctccggtgacacacattaacaccaactgctgtgggattggccagtgccacgcgtggtcacatggtctccccctgggtttcgcctctgttcctctttgcagttgtcctgtacagcgcgttcggctttccggaacccctgggcttttacaagcggggcaggccactgctctttcaaaggaggagggaggcagagggctgatggatcggtgaatttgctctgacactaggccttgagacctatggggtcattgtgtgctgcagcgaggccccgccggcctgaccagatgtggtgagcccatcctatgtcactcggagggggccagaatgggatctcaacgggagtccggagccacacagcaggcgtcctgaaactccccctccctcgctgggaatcggctcaagcaggtcctgaggacaggagccctgggggtttgggcctgtgacacgacgagacacccgcggcccccgctcccacgccgccccaaacaggacccaggacacagccgacgccggggcggcagcaggagcatc

SEQ ID NO: 3 - DNA, 26 bp, Artificial Sequence
  Chemically synthesized linker useful in the analysis of NBL2 repeat
ccctagcgatgcgttcgagcatcgct

SEQ ID NO: 4 - DNA, 26 bp, Artificial Sequence
  New and improved linker for Region 1 of NBL2 consensus sequence
ccctagcgatgcddddddgcatcgct

SEQ ID NO: 5 - DNA, 21 bp, Artificial Sequence
  Chemically synthesized primer useful in the analysis of NBL2 repeat
tttttgtgggtttgtgttagt

SEQ ID NO: 6 - DNA, 22 bp, Artificial Sequence
  Chemically synthesized linker useful in the analysis of NBL2 repeat
caaaaacatctttattcctcta

SEQ ID NO: 7 - DNA, 20 bp, Artificial Sequence
  Chemically synthesized primer useful in the analysis of NBL2 repeat
aygtggtttgggttaggtat

SEQ ID NO: 8 - DNA, 231 bp, Unknown
  subregion 2 of consensus sequence of NBL2
gagggctcctgctctgcaaagcctcggggactagtgtctaagacaatcgtgggaaccactgtgacctgagaaaccgctggygcttagcgtrtgcgcattggctgagccgactcacgctccactcctgacagataggctgcgtcccctttaaatatcgccatcgtcgcgcggcggccgcgatgctcctgctgccgcctcggtagcgtagcgatgcgttcgagcatcgttgcg

SEQ ID NO: 9 - DNA, 25 bp, Artificial Sequence
  Chemically synthesized linker AlwNI for subregion 2 of consensus sequence of NBL2
agcgatgcgttcgagcatcgctgcg

SEQ ID NO: 10 - DNA, 22 bp, Artificial Sequence
  Chemically synthesized forward primer for subregion 2 of NBL2 consensus sequence
gagggtttttgttttgtaaagt

SEQ ID NO: 11 - DNA, 20 bp, Artificial Sequence
  Chemically synthesized reverse primer for subregion 2 of NBL2 consensus sequence
tacccaaacactaccacaaa

SEQ ID NO: 12 - DNA, 22 bp, Artificial Sequence
  Chemically synthesized reverse primer for subregion 2 of NBL2 consensus sequence
ccacaaaaaaactcctactcta

SEQ ID NO: 13 - DNA, 25 bp, Unknown
  Improved AlwNI linker of subregion 2 of NBL2 consensus sequence
agcgatgcddddddgcatcgctgcg

(19) United States
(12) Patent Application Publication
(10) Pub. No.: US 2007/0087358 A1
(43) Pub. Date: Apr. 19, 2007

(54) Methods for diagnosing cancer based on DNA methylation status in NBL2

(76) Inventors: Melanie Ehrlich, Baltimore, MD (US); Rie Nishiyama, Ushiku (JP); Michelle Lacey, Durham, NC (US)

Correspondence Address: JONES DAY, 222 EAST 41ST ST, NEW YORK, NY, 10017, US

(21) Appl. No.: 11/255,232

(22) Filed: Oct. 19, 2005

(51) Int. Cl. C12Q 1/68

(52) U.S. Cl. 435/6

(57) ABSTRACT

The present invention relates to methods for diagnostic or prognostic assay for cancer based on analysis of altered methylation status at specific CpG dinucleotide sequences within the epigenetic marker, NBL2. The methods of the invention comprise determining the methylation status of a subregion of genomic CpG dinucleotide sequences within the DNA repeat, NBL2, in a sample of a subject and comparing the methylation status of the genomic CpG dinucleotide sequences in the sample to the methylation status of the genomic CpG dinucleotide sequences in a reference, wherein a difference in the methylation status of the genomic CpG dinucleotide sequences in the sample as compared to the reference indicates an association of the subject with cancer or cancer progression. The invention further relates to genomic DNA sequences that exhibit altered CpG methylation status in disease state as compared to normal state. The invention also provides nucleic acids, nucleic acid arrays and kits useful for practicing the methods of the present invention.

This invention was made with Government support under grant number CA81506 awarded by the National Institutes of Health. The United States Government has certain rights in the invention.

### 1. FIELD OF THE INVENTION

The present invention relates to methods for detecting or diagnosing cancer based on analysis of the methylation status at specific CpG dinucleotide sequences within the genomic target NBL2. The methods of the invention comprise determining the methylation status of a subset of genomic CpG dinucleotide sequences within the DNA repeat, NBL2, in a sample of a subject and comparing the methylation status of the genomic CpG dinucleotide sequences in the sample to the methylation status of the genomic CpG dinucleotide sequences in a reference genomic nucleic acid from a healthy subject, wherein a difference in the methylation status of the genomic CpG dinucleotide sequences in the sample as compared to the reference indicates an association of the subject with cancer or cancer progression. The invention further relates to genomic DNA sequences that exhibit altered CpG methylation status in a disease state as compared to a normal state. The invention also provides nucleic acids, nucleic acid arrays and kits useful for practicing the methods of the present invention.

### 2. BACKGROUND OF THE INVENTION

Cancer is characterized primarily by an increase in the number of abnormal cells derived from a given normal tissue, invasion of adjacent tissues by these abnormal cells, and lymphatic or blood-borne spread of malignant cells to regional lymph nodes and to distant sites (metastasis). Clinical data and molecular biological studies indicate that cancer is a multistep process that begins with minor preneoplastic changes, which may under certain conditions progress to neoplasia.

Pre-malignant abnormal cell growth is exemplified by hyperplasia, metaplasia, or most particularly, dysplasia (for review of such abnormal growth conditions, see Robbins, et al. (1976). Basic Pathology, 2d Ed., W.B. Saunders Co., Philadelphia, pp. 68-79.) The neoplastic lesion may evolve clonally and develop an increasing capacity for growth, metastasis, and heterogeneity, especially under conditions in which the neoplastic cells escape the host's immune surveillance (Roitt, et al. (1993). Immunology, 3rd ed., Mosby, St. Louis, pps. 17.1-17.12).

A marker-based approach to tumor identification and characterization promises improved diagnostic and prognostic reliability. Typically, the diagnosis of cancer requires histopathological proof of the presence of the tumor. In addition to diagnosis, histopathological examinations also provide information about prognosis and selection of treatment regimens. Prognosis may also be established based upon clinical parameters such as tumor size, tumor grade, the age of the patient, and lymph node metastasis. In clinical practice, accurate diagnosis of cancer is important because treatment options, prognosis, and the likelihood of therapeutic response all vary broadly depending on the diagnosis. Accurate prognosis, or determination of distant metastasis-free survival or overall survival could allow the oncologist and the patient to make treatment decisions.

Epigenetic information provides instructions on how, where, and when the genetic information should be used. Epigenetics is changes in the genome that do not involve changes in DNA sequence. One example is changes in DNA methylation. Alterations in DNA methylation have been recognized as one of the most common molecular alterations in human neoplasia. The first type of epigenetic change reported in human cancer was DNA hypomethylation. Feinberg, et al. (1983). Nature, 301, 89-92; Feinberg, et al. (1983). Biochem Biophys Res Commun, 111, 47-54; Gama-Sosa, et al. (1983). Nucleic Acids Res, 11, 6883-94. Subsequently, the opposite type of change, cancer-linked hypermethylation of CpG island-promoters of tumor suppressor genes, was shown to be crucial to downregulating expression of many alleles not inactivated by mutation. Jones, et al. (2002). Nat Rev Genet, 3, 415-28; Costello, et al. (2000). Nat. Genet., 24, 132-8. Although the functional importance of cancer-linked DNA hypomethylation is less well understood than that of hypermethylation, loss of DNA methylation is frequently observed in various cancers. Downregulating DNA methylation in some model systems increases tumor formation, while upregulating it in others does the same.

Both hypermethylation and hypomethylation of DNA have been observed in most tested cancers, but in different sequences (Narayan, et al. (1998). Int. J. Cancer, 77, 833-838; Santourlidis, et al. (1999). Prostate, 39, 166-174; Bariol, et al. (2003). Am. J. Pathol., 162, 1361-1371). Many specific gene regions become hypermethylated, and some other gene regions and many non-coding DNA repeats become hypomethylated during carcinogenesis (De Smet, et al. (1996). Proc. Natl. Acad. Sci. USA, 93, 7149-7153; Jones, et al. (2002). Nat. Rev. Genet., 3, 415-428; Ehrlich, (2002). Oncogene, 21, 5400-5413. Nonetheless, hypomethylation and hypermethylation in different parts of the genome in various cancers have been found not to be significantly associated with each other (Santourlidis, et al. (1999). Prostate, 39, 166-174; Ehrlich, et al. (2002). Oncogene, 21, 6694-6702; Erlich, et al., unpub. data). Therefore, cancer-linked DNA hypomethylation is not simply a response to cancer-linked hypermethylation nor vice versa.

It would, therefore, be beneficial to provide specific methods to use methylation pattern for diagnosing cancer in a subject. Such epigenetic changes may be found in many types of cancer and can, therefore, serve as potential markers for oncogenic transformation, provided that there is a reliable means for rapidly determining such epigenetic changes. The purpose of the present invention is to provide a method for determining epigenetic changes for the diagnosis of cancer, cancer therapeutic outcomes and survival of a subject. This method identifies subjects that have cancer and predicts which subjects are susceptible to cancer. Thus, early treatment may be implemented.

There is a need in the art for a sensitive clinically relevant diagnostic or prognostic assay for cell proliferative disorder, especially cancer, that is based, at least in part, on detection of variation in methylation status of CpG dinucleotide sequences, and that has a high percentage of diagnostic or prognostic accuracy.

Citation or discussion of a reference herein shall not be construed as an admission that such is prior art to the present invention.

### 3. SUMMARY OF THE INVENTION

The present invention harnesses the potential of genomic methylation of specific CpG dinucleotides as indicators of the presence of cancer in an individual and provides a reliable diagnostic and/or prognostic method applicable to cancer associated with altered methylation status of genomic CpG dinucleotides. Presently, there are no commercially available diagnostic and/or prognostic assays for the analysis of the methylation status of CpG dinucleotide sequence positions as markers for cancer.

The present invention is based on the identification of differentially methylated CpG dinucleotide positions within a nonsatellite tandem repeat in the genome, NBL2 (DMHD-1; CNIC; Y10752), for use as a reliable diagnostic, prognostic and/or staging marker for cancer. NBL2 has a high (C+G) content and a high ratio of (observed CpG)/(expected CpG) (60% and 0.67, respectively, for Y10752). NBL2 is found in BAC clone AC0128692, which contains 20 full-length and two partial copies of NBL2 with over 90% homology to one another and to Y10752 and U59100. Generally, for the methods provided by the invention, one or more CpG dinucleotide sequences are selected that are located within a subregion of the NBL2 genomic marker for determination of methylation status in the genomic DNA of a given tissue sample.

The present invention is directed to a method for detecting or diagnosing cancer in a subject, the method comprising: (a) determining the methylation status at one or more CpG dinucleotides of NBL2 in a biological sample obtained from said subject at one or more CpG dinucleotide sequences of an NBL2 sequence, and (b) comparing the methylation status of one or more CpG dinucleotide sequences of the NBL2 sequence in the sample to the methylation status from a reference sample at the corresponding one or more genomic CpG dinucleotide sequences, wherein a difference in the methylation status at one or more CpG dinucleotide sequences in the sample compared to the reference indicates a change in methylation status.

The present invention is directed to a method for detecting or diagnosing cancer in a subject, the method comprising: (a) determining the methylation status at one or more CpG dinucleotides of NBL2 of each strand of a double stranded genomic nucleic acid molecule in a biological sample obtained from said subject at one or more CpG dinucleotide sequences of an NBL2 sequence, and (b) comparing the methylation status of each strand of the double stranded genomic nucleic acid molecule at one or more CpG dinucleotide sequences of the NBL2 sequence in the sample to the methylation status of each strand of a double stranded genomic nucleic acid molecule from a reference sample at the corresponding one or more genomic CpG dinucleotide sequences wherein a difference in the methylation status of each strand of the double stranded genomic nucleic acid molecule at one or more CpG dinucleotide sequences in the sample compared to the reference indicates a change in methylation status.

The present invention is directed to a method wherein the methylation status of one or more CpG dinucleotide sequences is determined in a method comprising the steps of: (a) treating the genomic DNA with a bisulfite reagent; (b)amplifying a portion of the NBL2 sequence; and (c) determining the methylation status of the amplified sequence by pyrosequencing.

The present invention is directed to a method wherein the methylation status of one or more CpG dinucleotide sequences is determined in a method comprising the steps of: (a) digesting the genomic DNA from the sample with a methylation sensitive restriction enzyme; (b) ligating the genomic DNA to a linker; (c) denaturing the genomic DNA; (d) treating the genomic DNA with a bisulfite reagent; (e) heating the genomic DNA; (f) contacting the genomic DNA with an amplification enzyme and at least two primers that hybridizes to a nucleic acid molecule comprising a portion of the nucleotide sequence of SEQ ID NO:1 or 8, or is at least 80% identical to SEQ ID NO:1 or 8; and (g) determining the methylation status of one or more CpG dinucleotide sequence in the genomic DNA.

### 4. BRIEF DESCRIPTION OF DRAWINGS

FIGS. 1A-B illustrate the hairpin-bisulfite PCR genomic sequencing methodology. (A) Hairpin-bisulfite PCR of the NBL2 repeat is shown schematically. The covalently linked upper and lower strands (not to scale) are diagrammed as a hairpin to illustrate their complementarity before bisulfite deamination of all unmethylated C residues. ^{m}C, 5-methylcytosine. The recognition site for BsmAI is in italics, and its cleavage specificity is shown on the right. (B) Examples of discrimination between different methylated configurations of CpG dyads and C-to-T changes in a genomic sequence (e.g., polymorphisms) by sequencing hairpin-bisulfite PCR products. Note that part of one strand of each molecular clone is depicted in the hairpin configuration to align CpG positions that were part of a genomic dyad. Positions 1 and 2 in (B) correspond to positions 1 and 2 in (A).

FIG. 2 shows the location of the hairpin bisulfite-sequenced portion of NBL2 and restriction maps. The gray band denotes the subregion used for hairpin-bisulfite PCR. The positions of the restriction sites relative to the single NotI site are shown for the 1.4-kb NBL2 sequence GenBank Y10752. Note that there is only ˜93% homology between NBL2 in Y10752 and the 20 tandem copies of NBL2 in AC018692 and among the 20 copies in AC018692. A schematic of the hairpin product, with the linker at the right is given at the bottom of the figure.

FIG. 3 shows the hairpin-bisulfite PCR genomic sequencing result from subregion 1 of NBL2 for normal tissues and ovarian carcinomas. Hairpin-bisulfite PCR-derived genomic sequences are shown for each clone from three somatic controls and five ovarian carcinomas, but each observed epigenetic pattern for a given sample is illustrated only once. The most abundant pattern for each sample is boxed. M/M, a symmetrically methylated CpG dyad; U/U, a symmetrically unmethylated CpG dyad; M/U and U/M, the two orientations of hemimethylated CpG dyads; -, no CpG was present at that site due to sequence variation; NA, methylation could not be analyzed due to sequencing mistakes. At the top of each column, the following are given: the CpG site number (pink highlighting for sites always M/M in somatic controls and yellow for sites never M/M in somatic controls), nucleotide position within the sequenced region (beginning immediately after the forward 20-base primer for the second round of PCR), and overlapping CpG methylation-sensitive restriction sites (Hpy4 is the abbreviation for HpyCH4IV). The Southern Blot-derived HhaI methylation score (Nishiyama et al., 2005) is also stated. There were three CpG positions that were much less frequently present in these samples due to much sequence variation. Note that the somatic control tissues came from three individuals.

FIG. 4 shows genomic sequencing results, as in FIG. 3, for unmethylated NBL2 plasmid, in vitro-methylated (at CpG's) NBL2 plasmid, normal sperm DNA, and five Wilms tumors.

FIGS. 5A-C show a comparison of methylation in somatic control tissues (brain, spleen, and lung), ovarian carcinomas, and Wilms tumors. (A) Cartoon illustrating to scale the positions of the CpG sites in the hairpin-bisulfite sequenced region and their methylation status in the somatic controls. The 7 CpG's that were always M/M and the 2 CpG's that were either always U/U (CpG14) or usually U/U and occasionally hemimethylated (CpG6) are shown above the horizontal line. The variably methylated CpG's are shown as diamonds below the line. The filled-in circle below the line represents CpG13, which was always methylated when present, but often not present due to germline sequence variation. (B) and (C) show the overall change in methylation in five ovarian carcinomas and five Wilms tumors at CpG's that were either always M/M or never M/M in somatic controls. The % change in methylation at CpG2, 3, 5, 8, 10, 11, and 12 is the percentage of cancer clones with hypomethylation (loss of M/M status) at that position; for CpG6 and 14, it is the percentage of cancer clones with hypermethylation (gain of M/M status).

FIGS. 6A-D are representative Southern Blot analysis of NBL2 hyper- and hypomethylation in cancer DNAs. Ovarian carcinoma, Wilms tumor, and control DNAs were digested with the indicated CpG methylation-sensitive enzymes and probed with the 1.4-kb NBL2 sequence. The brackets in (A) and (C) indicate the separate hypermethylated and hypomethylated fractions of NBL2 repeats in OvCaD and in OvCaE although the hypomethylated repeats were more prominent, especially for HhaI digests. Different exposures from the same blot were used for these panels. Note with respect to the restriction map of FIG. 2, that there is appreciable sequence variation in NBL2, and at least three sequences containing the whole repeat are available (GenBank Y10752), U59100, and AC018692). Other sequences which are suitable for use in the method of the present invention are as follows: AJ338130, AL935212, AL118524, AL627230, AL391987, AC146073, AL953889, AL121762, AJ338193, AL591926, AL773537, AJ343471, AJ335302, BX005037, AL162731, AJ336724, AJ337004, AJ343469, AL450124, or AL390198.

FIGS. 7A-E are an analysis of methylation in immunodeficiency, centromeric region, facial anomalies syndrome (“ICF”) and control LCLs. (A-D), Southern blot analysis. (E). Genome sequencing results. The ICF LCLs were ICF B, C, and S and the control LCLs were maternal B, maternal C, and paternal C, respectively, from phenotypically normal parents of ICF patients (Ehrlich, et al. (2001). Hum. Mol. Genet., 10, 2917-2931; Tuck-Muller, et al. (2000). Cytogenet. Cell Genet., 89, 121-128). The somatic control tissues for (A) and (B) were brain, lung, and heart; for (C), lung and spleen; and for (D), brain, lung, and spleen. Spm, normal sperm.

FIG. 8 is a map of methyl-CpG sensitive restriction sites in NBL2. Numbers in parentheses are the average number of the sites per monomer from existing DNA sequence information. Numbers above the bars are the positions of the sites and those below the bars are the size (bp) of the digested fragments. Subregion 1 and subregion 2 are the subregions amplified for the hairpin bisulfite sequencing. The map is shown for the Genbank NBL2 sequence Y10752, beginning at the single Not1 site. There is about 93% sequence identity between NBL2 in Y10752 and the 20 tandem copies of NBL2 in AC018692 and among the 20 copies in AC018692. A schematic of the hairpin product is given at the bottom of FIG. 8.

FIG. 9 is a consensus sequence of subregion 1 of NBL2. The sequence starts with a forward primer F2-2 (underlined) to the end of the linker (double underlined). CpG dinucleotide sequences useful for the present invention are identified as CpG1, CpG2, CpG3, CpG4, CpG5, CpG6, CpG7, CpG8, CpG9, CpG10, CpG11, CpG12, CpG13, and CpG14.

FIG. 10 is a consensus sequence of subregion 2 in NBL2. The sequence starts with a forward primer to the end of the A1wNI linker. CpG dinucleotide sequences useful for the present invention are underlined.

FIG. 11 shows the hairpin-bisulfite PCR genomic sequencing results from subregion 2 of NBL2 for normal tissues and ICF.

### 4.1 SEQUENCES

Below is a brief summary of the sequences presented in the accompanying sequence listing, which is incorporated by reference herein in its entirety:

SEQ ID NO:1 is a nucleotide sequence of a Region 1 from the forward primer F2-2 through the end of the linker of NBL2.

SEQ ID NO:2 is a nucleotide sequence of NBL2 consensus sequence (GenBank accession No. AC0128692.

SEQ ID NO:3 is a nucleotide sequence of a linker that is useful in the method of the present invention.

SEQ ID NO:4 is a nucleotide sequence of a linker that is useful in the method of the present invention.

SEQ ID NO:5 is a nucleotide sequence of a primer that is useful in the method of the present invention.

SEQ ID NO:6 is a nucleotide sequence of a linker that is useful in the method of the present invention.

SEQ ID NO:7 is a nucleotide sequence of a primer that is useful in the method of the present invention.

SEQ ID NO:8 is a nucleotide sequence of consensus subregion 2 of NBL2 with a forward primer sequence and an A1wNI linker sequence.

SEQ ID NO:9 is a nucleotide sequence of an A1wNI linker for subregion 2 of NBL2.

SEQ ID NO:10 is a nucleotide sequence of forward primer for subregion 2 of NBL2.

SEQ ID NO:11 is a nucleotide sequence of reverse primer for subregion 2 of NBL2.

SEQ ID NO:12 is a nucleotide sequence of reverse primer for subregion 2 of NBL2.

SEQ ID NO:13 is a nucleotide sequence of an A1wNI linker for subregion 2 of NBL2.

### 4.2 DEFINITIONS

As used herein, the term “methylation status” refers to the presence or absence of 5-methylcytosine at one or a more CpG dinucleotides within a DNA sequence.

As used herein, the term “methylation pattern” means the presence or absence of 5-methylcytosine at two or more CpG dinucleotides. In general, methylation status of two or more CpG dinucleotides forms a methylation pattern.

As used herein, the term “hypermethylation” refers to the methylation status corresponding to an increased presence of 5-methylcytosine at one or more CpG dinucleotides within a DNA sequence of a test DNA sample, relative to the amount of 5-methylcytosine found at corresponding CpG dinucleotides within a normal control DNA sample.

As used herein, the term “hypomethylation” refers to the methylation status corresponding to a decreased presence of 5-methylcytosine at one or more CpG dinucleotides within a DNA sequence of a test DNA sample, relative to the amount of 5-methylcytosine found at corresponding CpG dinucleotides within a normal control DNA sample.

As used herein, the term “hemi-methylation”, “hemimethylation” or “asymmetric methylation” refers to the methylation status of a palindromic CpG methylation site, where only a single cytosine in one of the two CpG dinucleotide sequences of the palindromic CpG methylation site is methylated. This is denoted as U/M, or M/U.

As used herein, the term “CpG dinucleotide of NBL2” means a dinucleotide sequence of CG in the NBL2 sequence or the complement of the NBL2 sequence.

As used herein, the term “each strand of the double-stranded nucleic acid molecule” means the two complementary strands of nucleic acids that forms the double-helix DNA molecule.

As used herein, the term “subregion of NBL2” means a DNA fragment of about 50-100 nucleic acids, 100-200 nucleic acids, 200-300 nucleic acids, 300-400 nucleic acids, 400-500 nucleic acids, 500-600 nucleic acids, 600-700 nucleic acids, 700-800 nucleic acids, 800-900 nucleic acids, 900-1,000 nucleic acids, 1,000-1,200 nucleic acids, 1,200-1,300 nucleic acids in length that lies within the NBL2 genomic sequence or a nucleic acid having a nucleotide sequence of SEQ ID NO: 2 or at least 80% identical to SEQ ID NO: 2. In a preferred embodiment, the “subregion of NBL2” is at nucleotide position 1-172, 172-372, 372-572, 572-772, 772-972, 972-1172, or 1172-1400 of SEQ ID NO: 2.

As used herein, the term “NBL2” in the context of a nucleic acid refers to a nucleic acid that comprises the nucleotide sequence of GenBank accession numbers Y10752, U59100, SEQ ID NO:2 or a nucleic acid that is at least 70%, at least 75%, at least 80%, at least 85%, at least 90%, at least 95% or at least 99% identical to in GenBank accession numbers Y10752, U59100 or SEQ ID NO:2. In other specific embodiments, NBL2 comprises the nucleotide sequence of GenBank accession numbers AJ338130, AL935212, AL118524, AL627230, AL391987, AC146073, AL953889, AL121762, AJ338193, AL591926, AL773537, AJ343471, AJ335302, BX005037, AL162731, AJ336724, AJ337004, AJ343469, AL450124, or AL390198. In a specific embodiment, the NBL2 is a tandem NBL2 array as found in BAC clone (AC018692). In specific embodiments, the NBL2 is on chromosome 13, 14, 15, 21, 9 or Y. In specific embodiments, the NBL2 is in 9q21 or 9p11 contigs, NT078064, NT078066, NT078077, NT078051, NJ078053 or NT086759 from GenBank.

As used herein, the term “stringent condition” refers to hybridization and washing conditions under which nucleotide sequences having at least 70%, at least 75%, at least 80%, at least 85%, at least 90%, or at least 95% identity to each other will detectably hybridize to each other. Such hybridization conditions are described in, for example but not limited to, Current Protocols in Molecular Biology, John Wiley & Sons, N.Y. (1989), 6.3.1-6.3.6.; Basic Methods in Molecular Biology, Elsevier Science Publishing Co., Inc., N.Y. (1986), pp. 75-78, and 84-87; and Molecular Cloning, Cold Spring Harbor Laboratory, N.Y. (1982), pp. 387-389, and are well known to those skilled in the art. A preferred, non-limiting example of stringent hybridization conditions is hybridization in 6× sodium chloride/sodium citrate (SSC), 0.5% SDS at about 68° C. followed by one or more washes in 2×SSC, 0.5% SDS at room temperature. Another preferred, non-limiting example of stringent hybridization conditions is hybridization in 6×SSC at about 45° C. followed by one or more washes in 0.2×SSC, 0.1% SDS at about 50-65° C. Yet another preferred, non-limiting example of stringent hybridization conditions is to employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C.; or to employ 50% formamide, 5×SSC (0.75 M NaCl, 0.075 M Sodium pyrophosphate, 5× Denhardt's solution, sonicated salmon sperm DNA (50 μg/ml), 0.1% SDS, and 10% dextran sulfate at 42° C., with washes at 42° C. in 0.2×SSC and 0.1% SDS.

To determine the percent identity of two nucleotide sequences, the sequences are aligned for optimal comparison purposes (e.g., gaps can be introduced in the sequence of a first nucleotide sequence for optimal alignment with a second nucleotide sequence). The nucleotide positions are then compared. When a position in the first sequence is occupied by the same nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin, et al. (1990). Proc. Natl. Acad. Sci. U.S.A., 87, 2264-2268, modified as in Karlin, et al. (1993). Proc. Natl. Acad. Sci. U.S.A., 90, 5873-5877. Such an algorithm is incorporated into the NBLAST and XBLAST programs of Altschul, et al. (1990). J. Mol. Biol., 215, 403. BLAST nucleotide searches can be performed with the NBLAST nucleotide program parameters set, e.g., for score=100, wordlength=12 to obtain nucleotide sequences homologous to a nucleic acid molecules of the present invention. To obtain gapped alignments for comparison purposes, Gapped BLAST can be utilized as described in Altschul, et al. (1997). Nucleic Acids Res., 25, 3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4: 11-17. Such an algorithm is incorporated in the ALIGN program (version 2.0) which is part of the GCG sequence alignment software package.

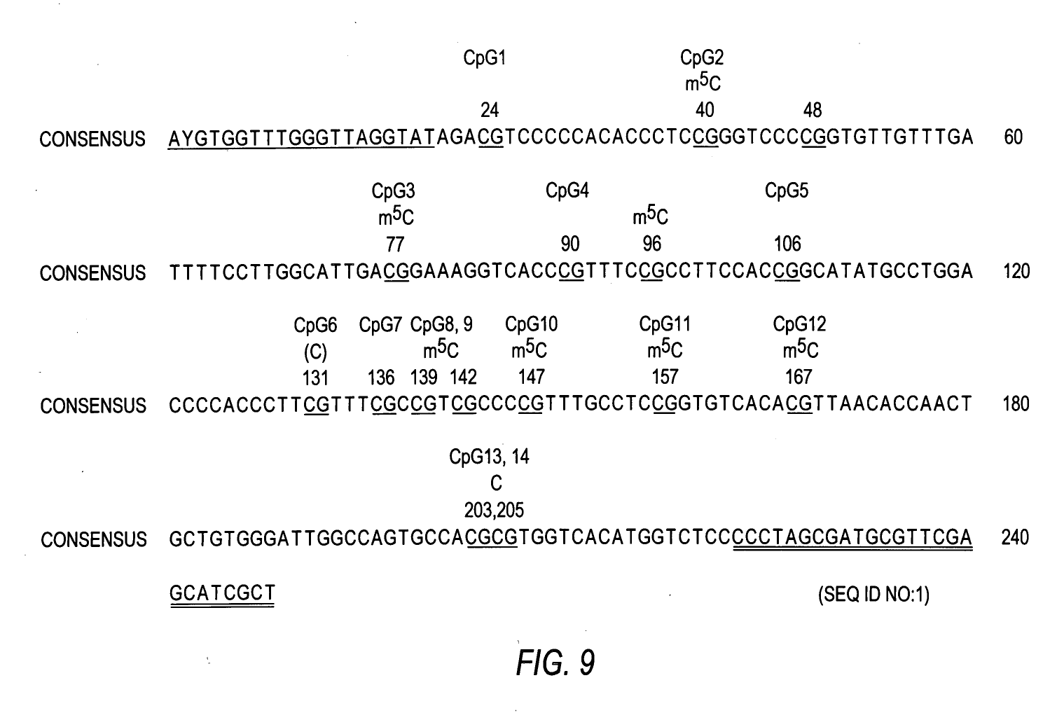

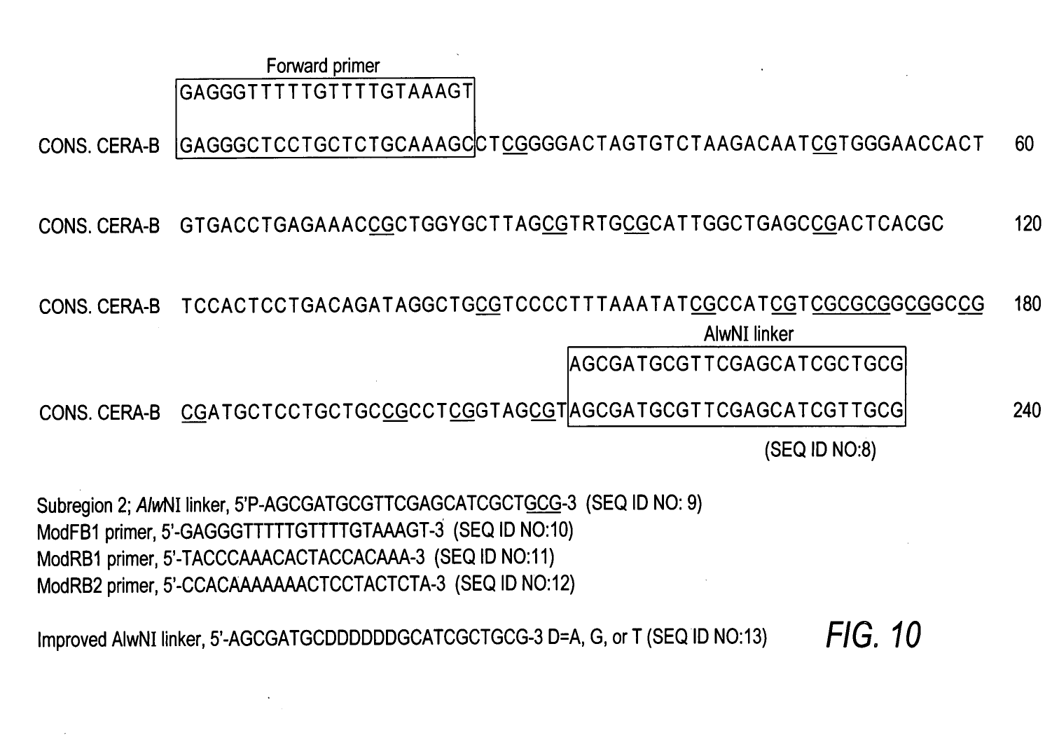

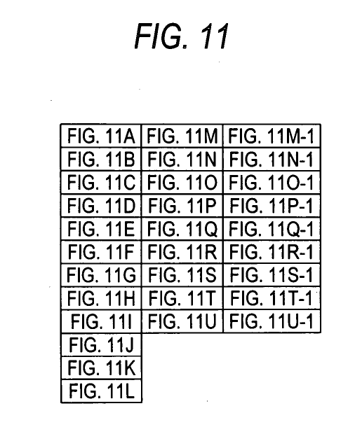

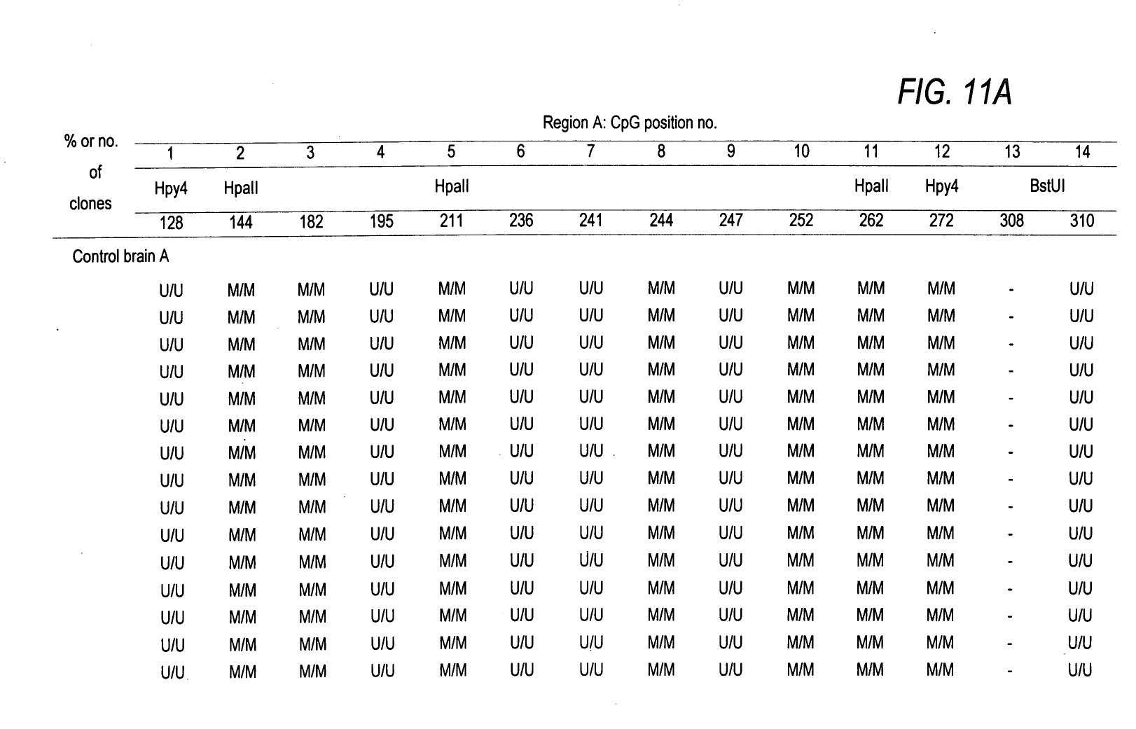

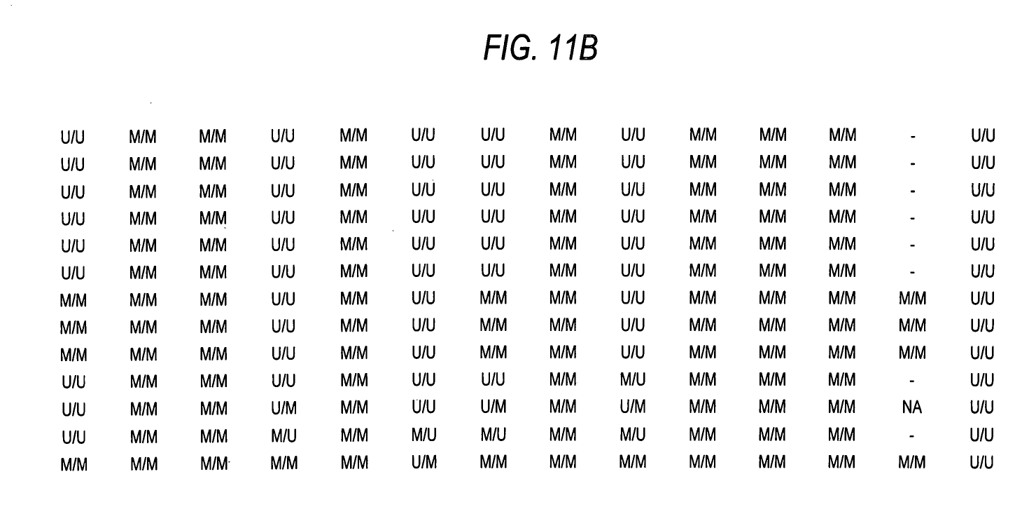

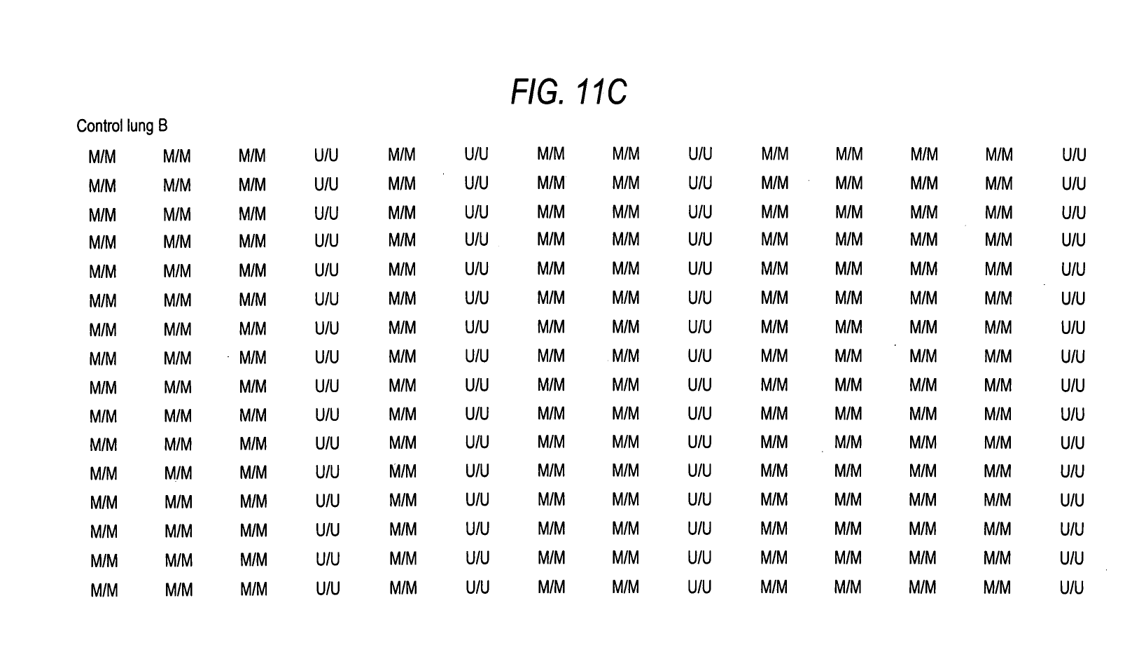

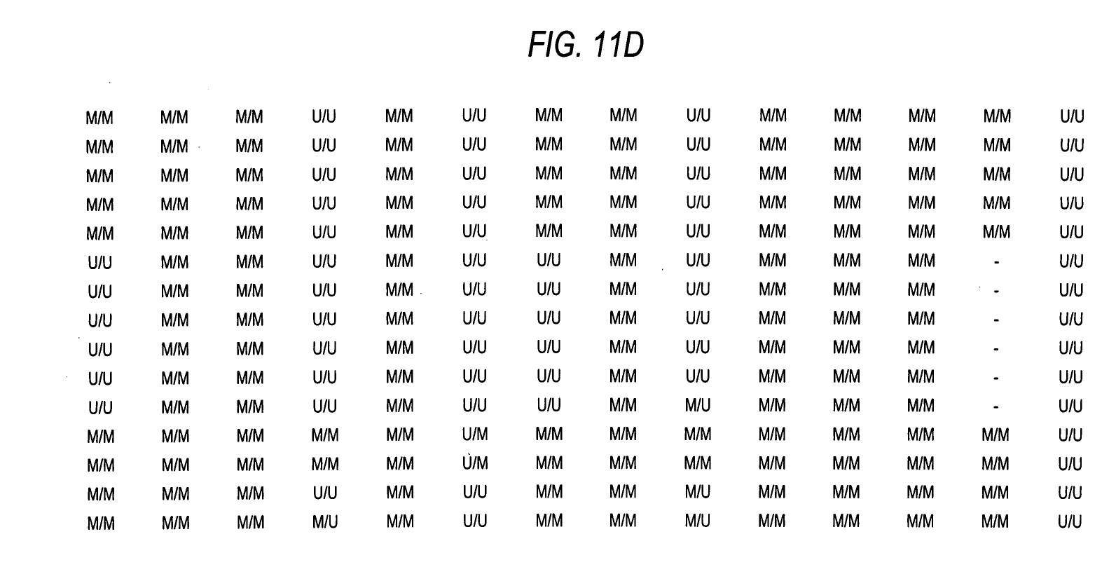

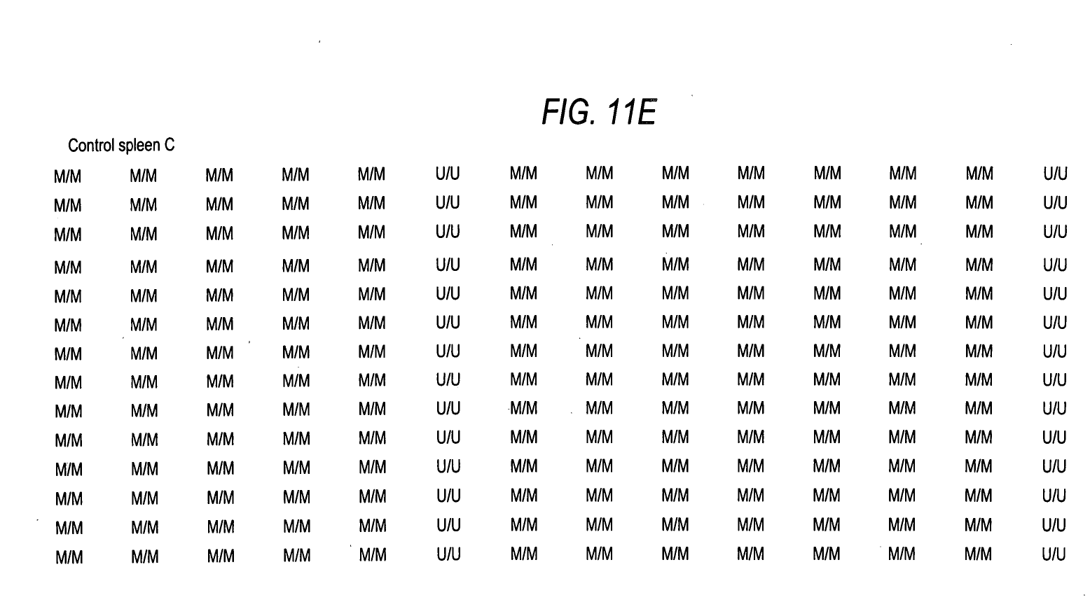

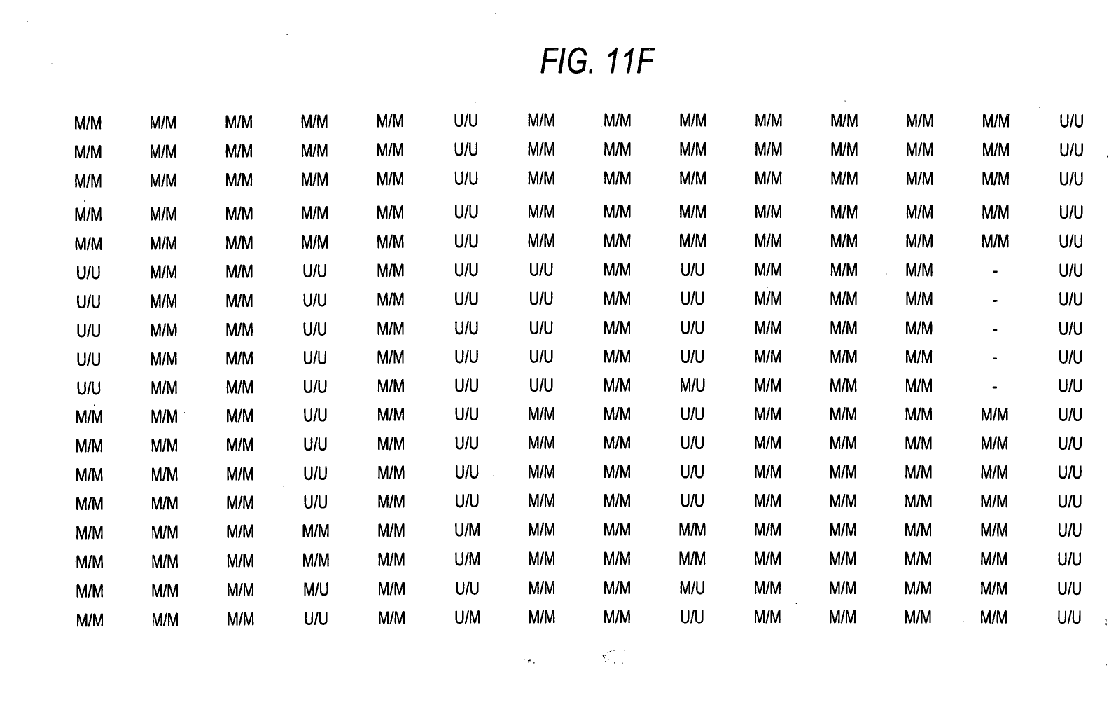

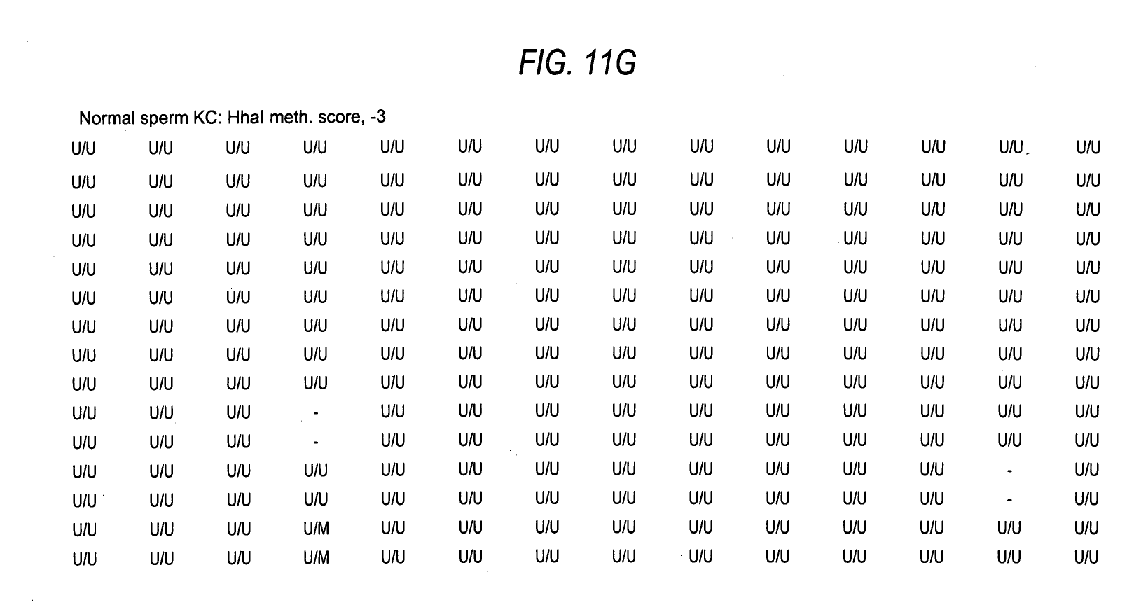

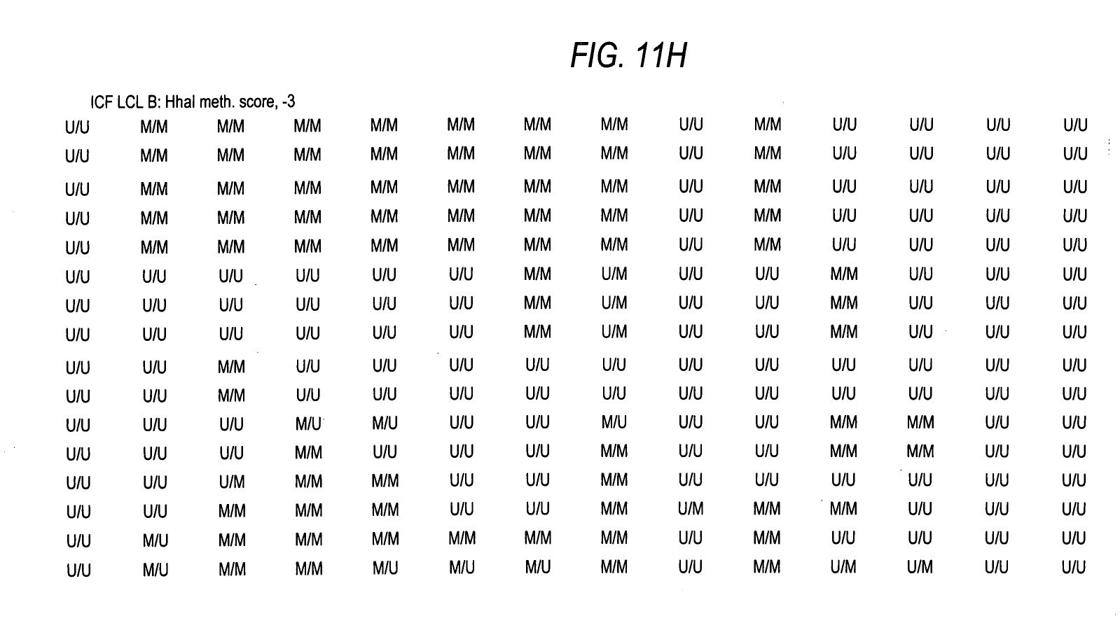

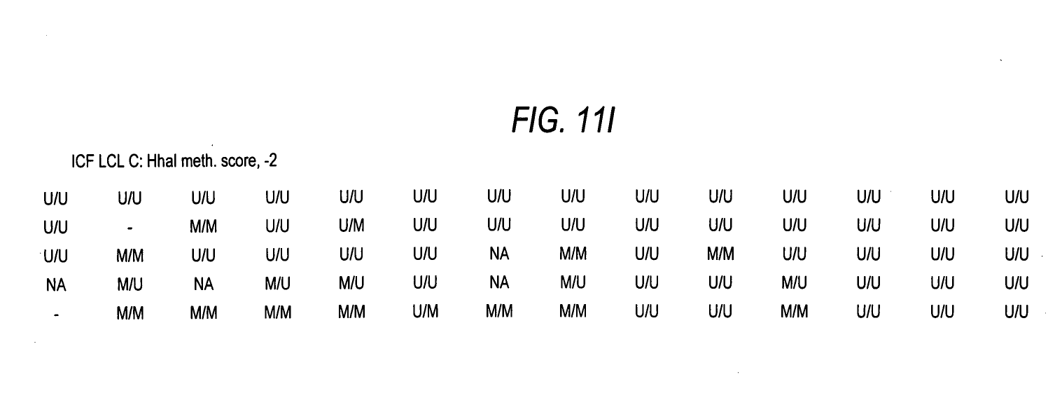

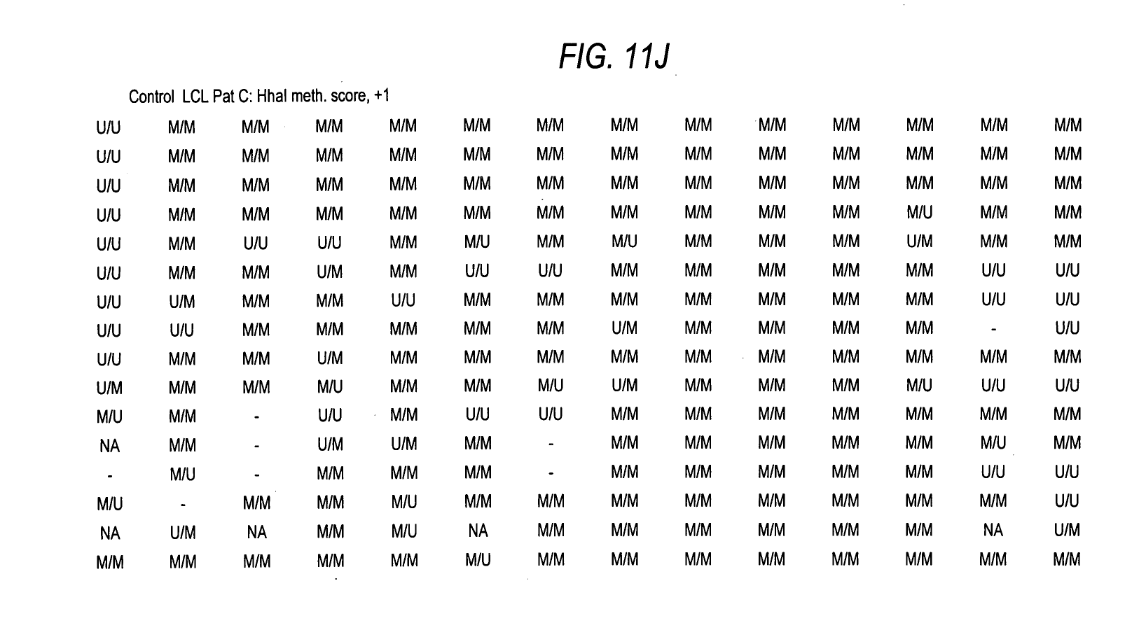

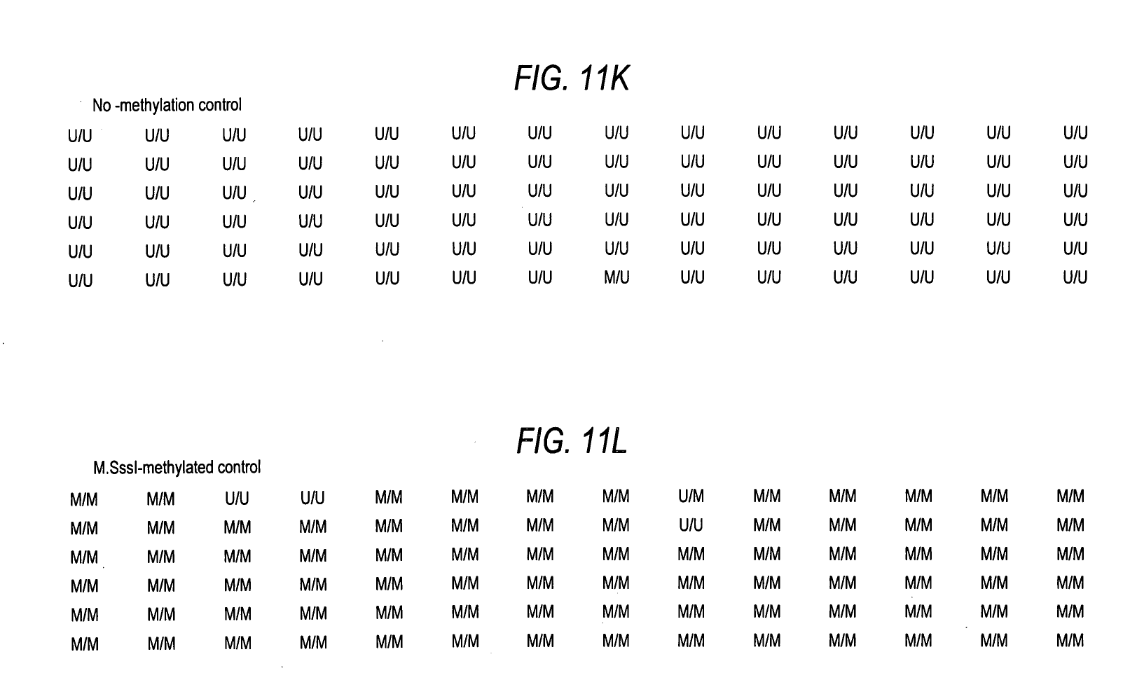

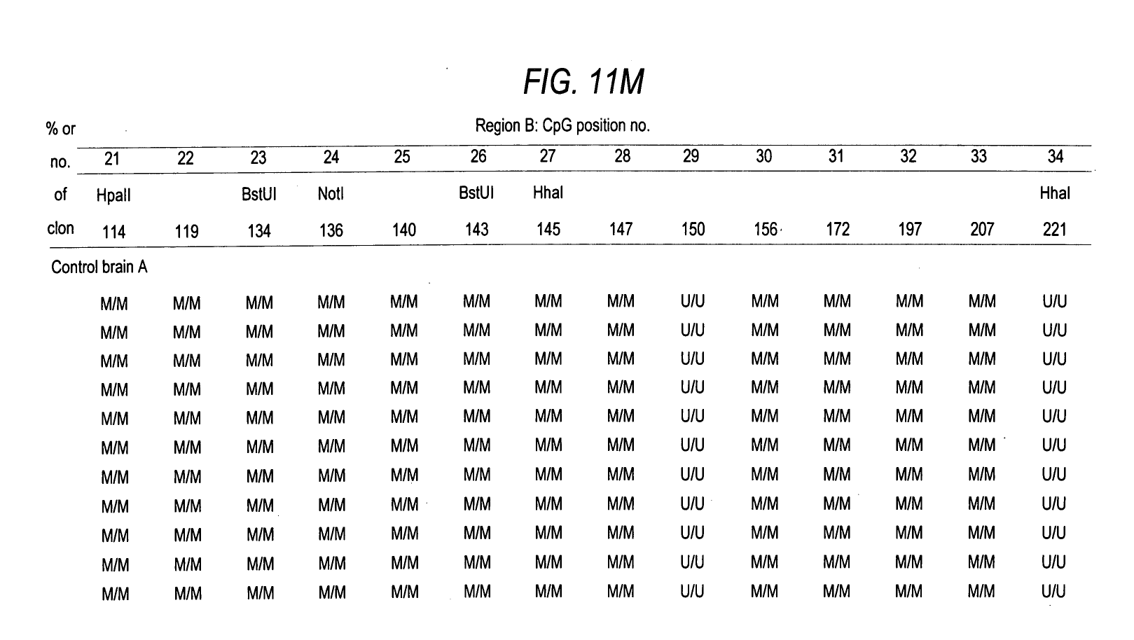

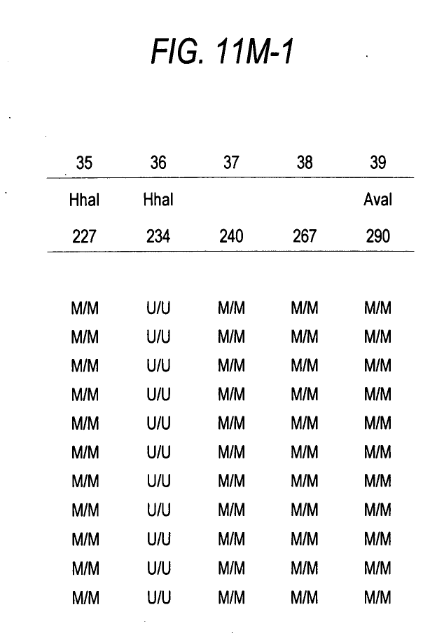

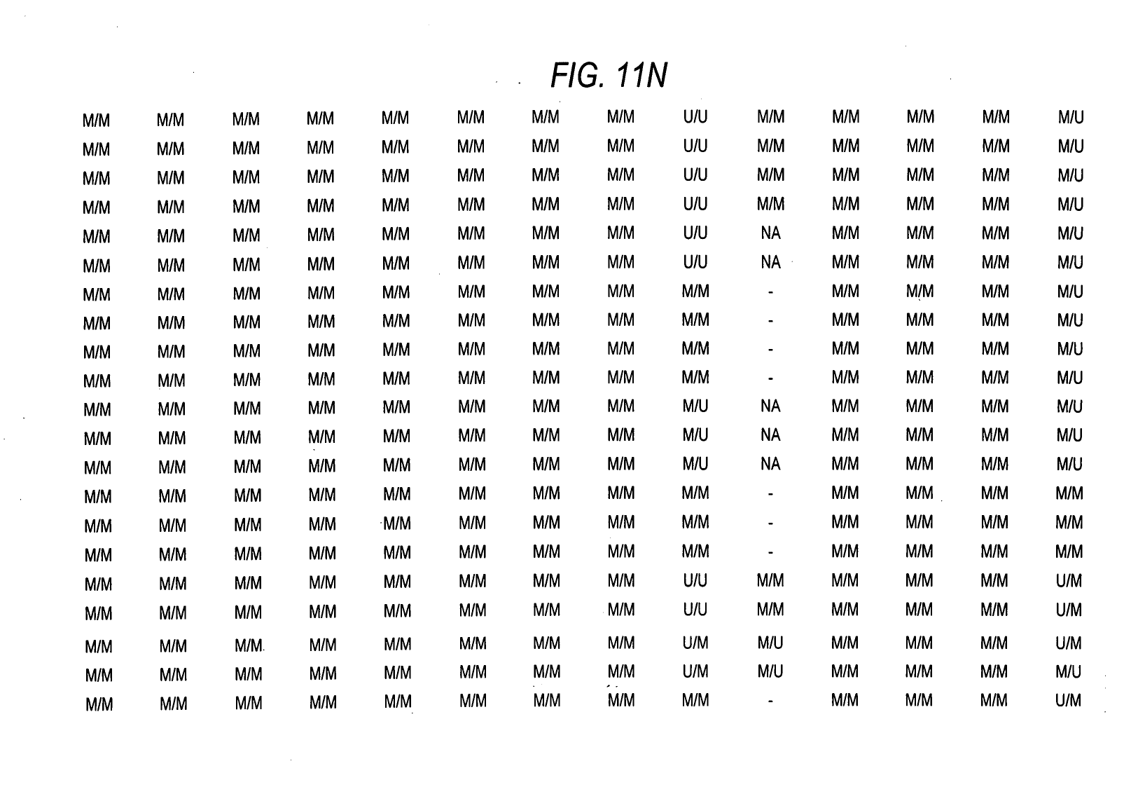

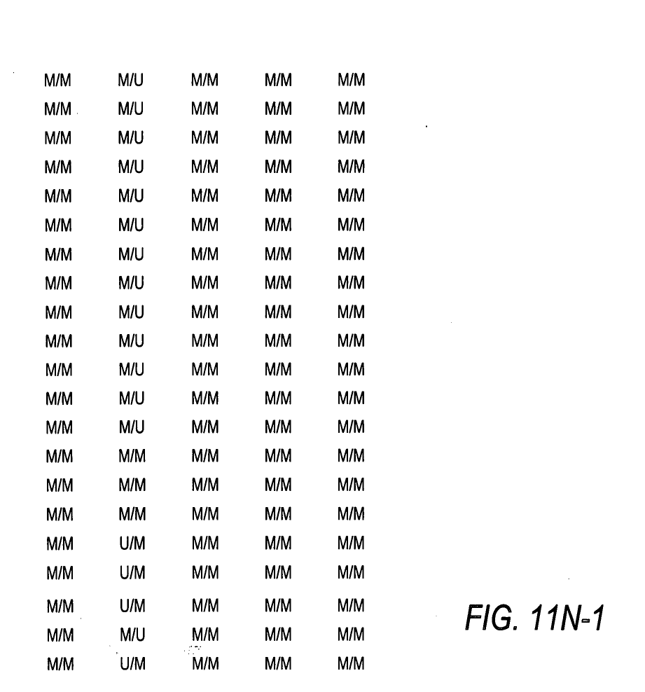

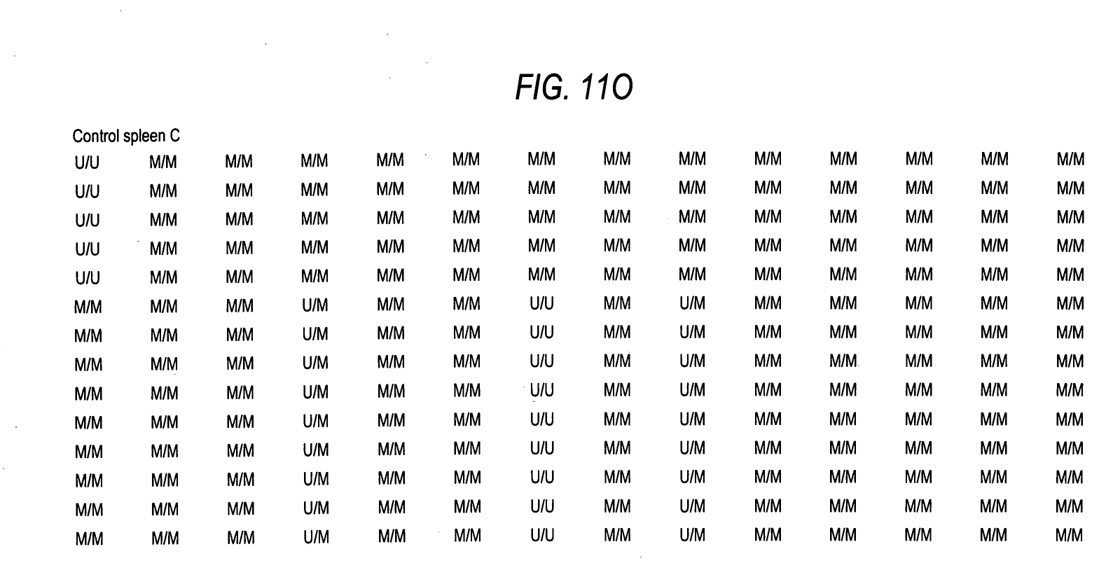

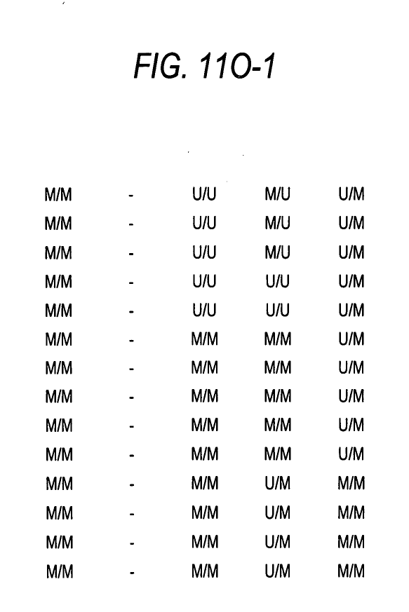

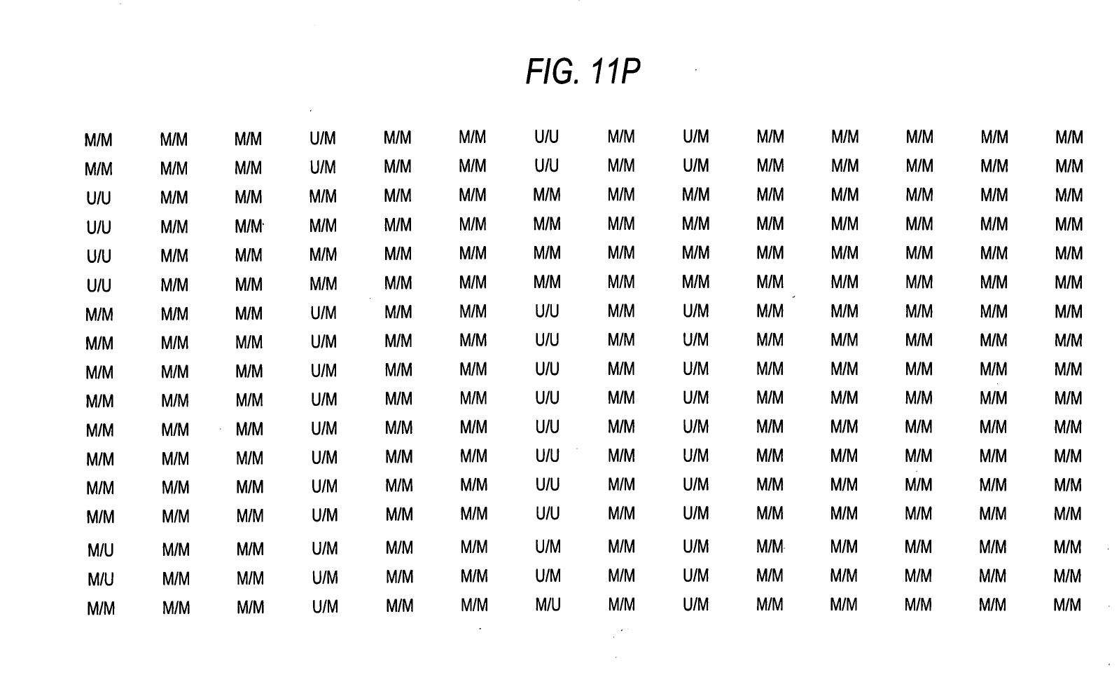

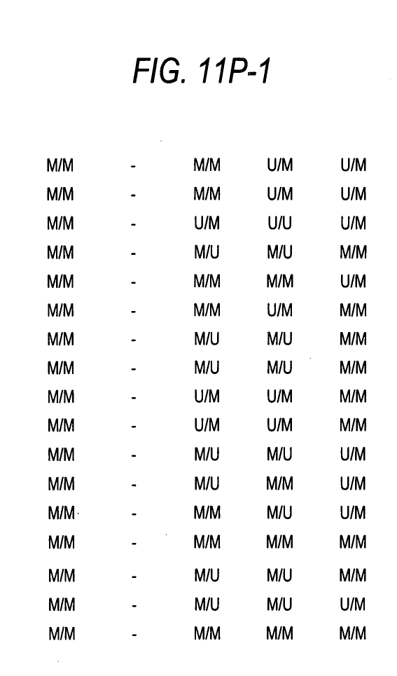

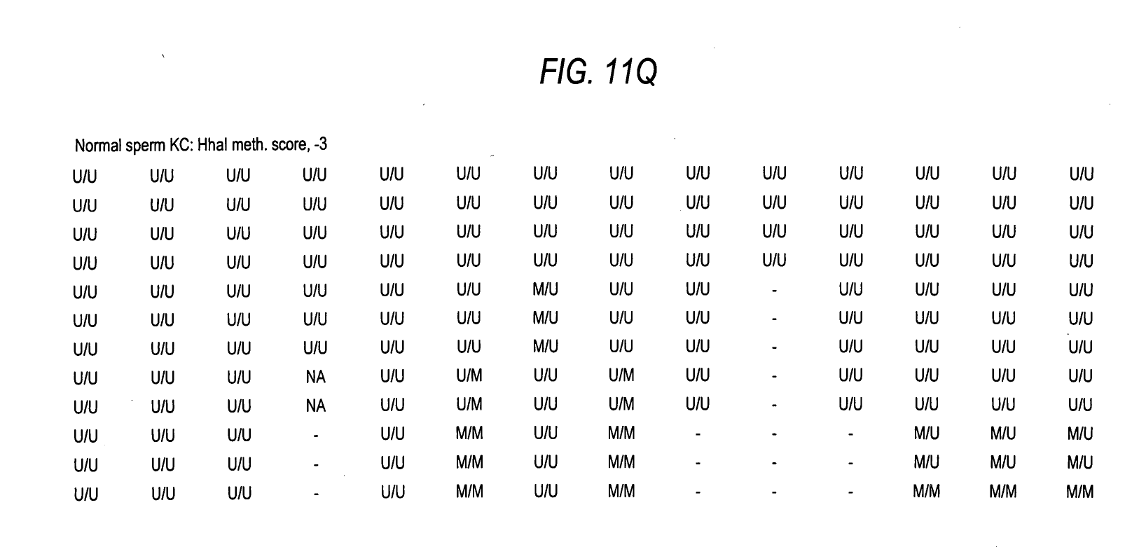

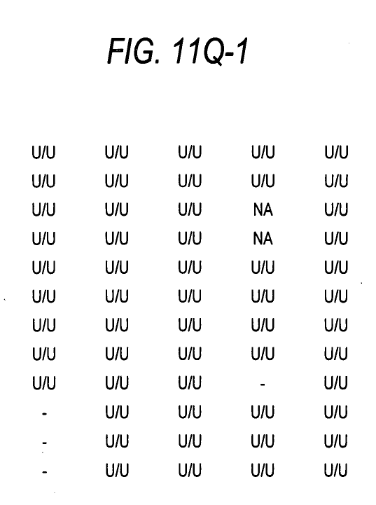

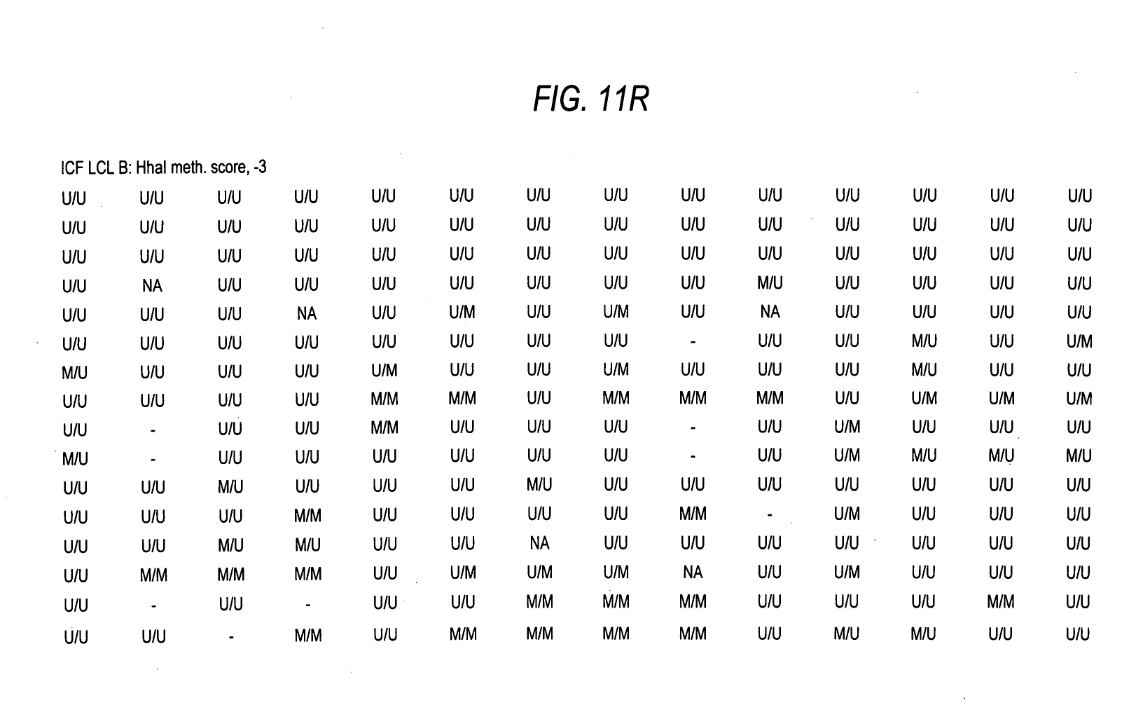

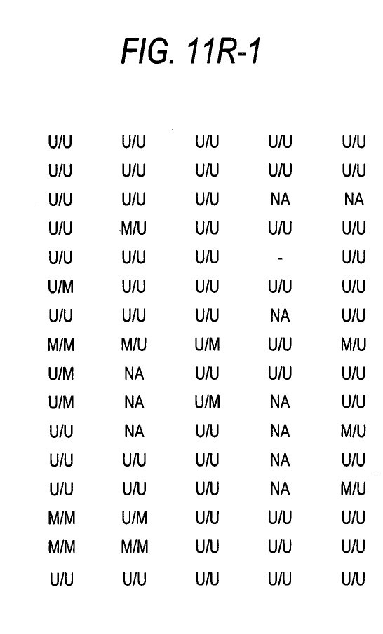

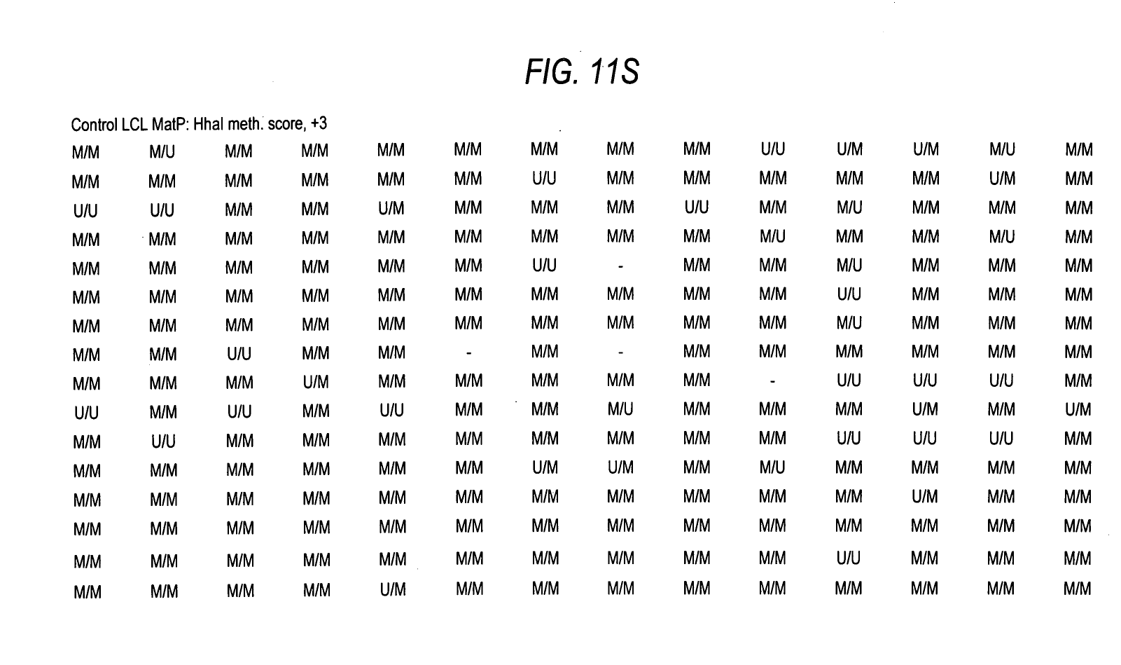

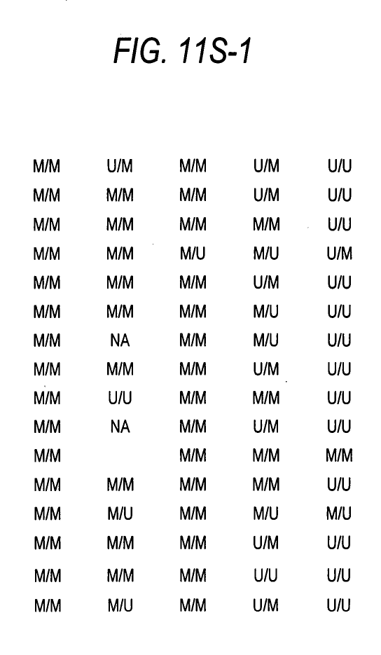

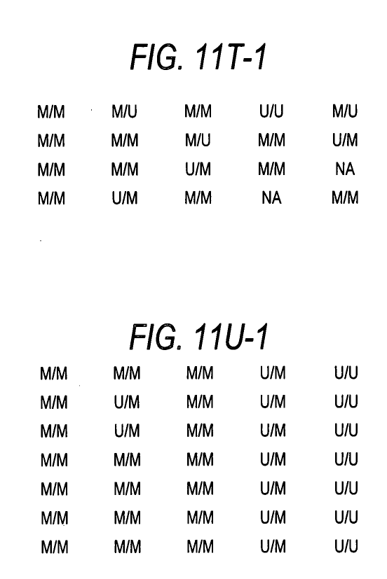

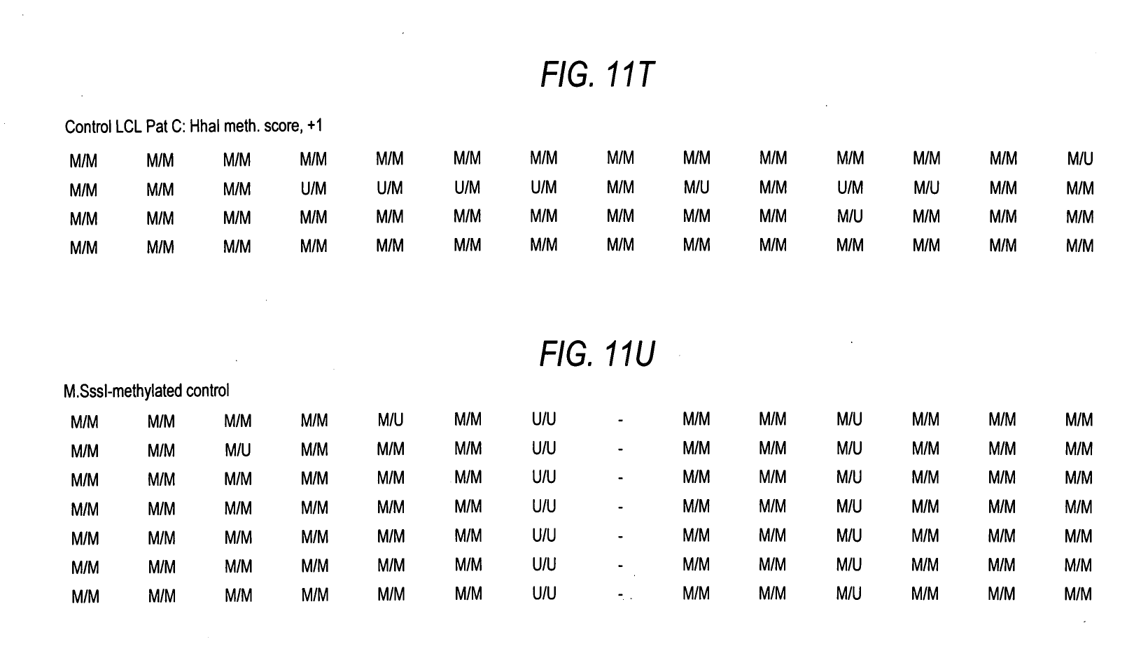

As used herein, the terms “CpG1”, “CpG2”, “CpG3”, “CpG4”, “CpG5”, “CpG6”, “CpG7”, “CpG8”, “CpG9”, “CpG10”, “CpG11”, “CpG12”, “CpG13”, and “CpG14” mean a dinucleotide sequence at a particular position within a subregion of the NBL2. In specific embodiments, the CpG dinucleotides have the following nucleotide positions on the consensus sequence, SEQ ID NO:1, as shown in FIG. 9: CpG1=24, 25; CpG2=40, 41; CpG3=77, 78; CpG4=90, 91; CpG5=106, 107; CpG6=131, 132; CpG7 =136, 137; CpG8=139, 140; CpG9=142, 143; CpG10=147, 148; CpG11=157, 158; CpG12=167, 168; CpG13=203, 204; CpG14=205, 206. Since the repeats are highly homologous to each other in an alignment of the repeats, one can determine the corresponding nucleotide position of a CpG dinucleotide in a subregion of a NBL2 repeat that is homologous to the consensus sequence. In other specific embodiment, the CpG dinucleotides have the following nucleotide positions on a consensus sequence of SEQ ID NO: 8 as shown in FIG. 10. CpG21=25, 26; CpG22-48, 49; CpG23=75, 76; CpG24=88, 89; CpG25=94, 95; CpG26=108, 109; CpG27=140, 141; CpG28=156, 157; CpG29 =62, 63; CpG30=165, 166; CpG31=167, 168; CpG32=169, 170; CpG33=172, 173; CpG34=176, 177; CpG35=178, 179; CpG36=193, 194; CpG37=198, 199. In a preferred embodiment, the subregion comprises a nucleotide sequence of SEQ ID NO:1 or a nucleotide sequence that is at least 80% identical to SEQ ID NO:1.

As used herein, the terms “nucleic acids” and “nucleotide sequences” include DNA molecules (e.g., cDNA or genomic DNA), RNA molecules (e.g., mRNA), combinations of DNA and RNA molecules or hybrid DNA/RNA molecules, and analogs of DNA or RNA molecules. Such analogs can be generated using, for example, nucleotide analogs, which include, but are not limited to, inosine or tritylated bases. Such analogs can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions, but preferably is double-stranded DNA.

As used herein, the term “diagnosis” refers to a process of determining if an individual is afflicted with cancer or for determining the grade or stage of cancer. In this context, “diagnosis” refers to a process whereby one increases the likelihood that an individual is properly characterized as being afflicted with a cancer or a grade or stage of cancer (“true positive”) or is properly characterized as not being afflicted with cancer or a grade or stage of cancer (“true negative”) while minimizing the likelihood that the individual is improperly characterized as being afflicted with cancer or a grade or stage or cancer (“false positive”) or improperly characterized as not being afflicted with cancer or a grade or stage of cancer (“false negative”).

As used herein, the term “neoplastic” refers to a disease involving cells that have the potential to metastasize to distal sites. Neoplastic cells acquire a characteristic set of functional capabilities during their development, albeit through various mechanisms. Such capabilities include evading apoptosis, self-sufficiency in growth signals, insensitivity to anti-growth signals, tissue invasion/metastasis, limitless replicative potential, and sustained angiogenesis. Thus, “non-neoplastic” means that the condition, disease, or disorder does not involve cancer cells.

As used herein, the term “neoplastic cell” refers to any cell that is transformed such that it proliferates without normal homeostatic growth control. Such cells can result in a benign or malignant lesion of proliferating cells. Such a lesion can be located in a variety of tissues and organs of the body. Exemplary types of cancers from which a neoplastic cell can be derived are set forth infra.

As used herein, the term “cancer” refers to a disease involving cells that have the potential to metastasize to distal sites. Cancer cells acquire a characteristic set of functional capabilities during their development, albeit through various mechanisms. Such capabilities include evading apoptosis, self-sufficiency in growth signals, insensitivity to anti-growth signals, tissue invasion/metastasis, limitless replicative potential, and sustained angiogenesis. The term “cancer cell” is meant to encompass both pre-malignant and malignant cancer cells.

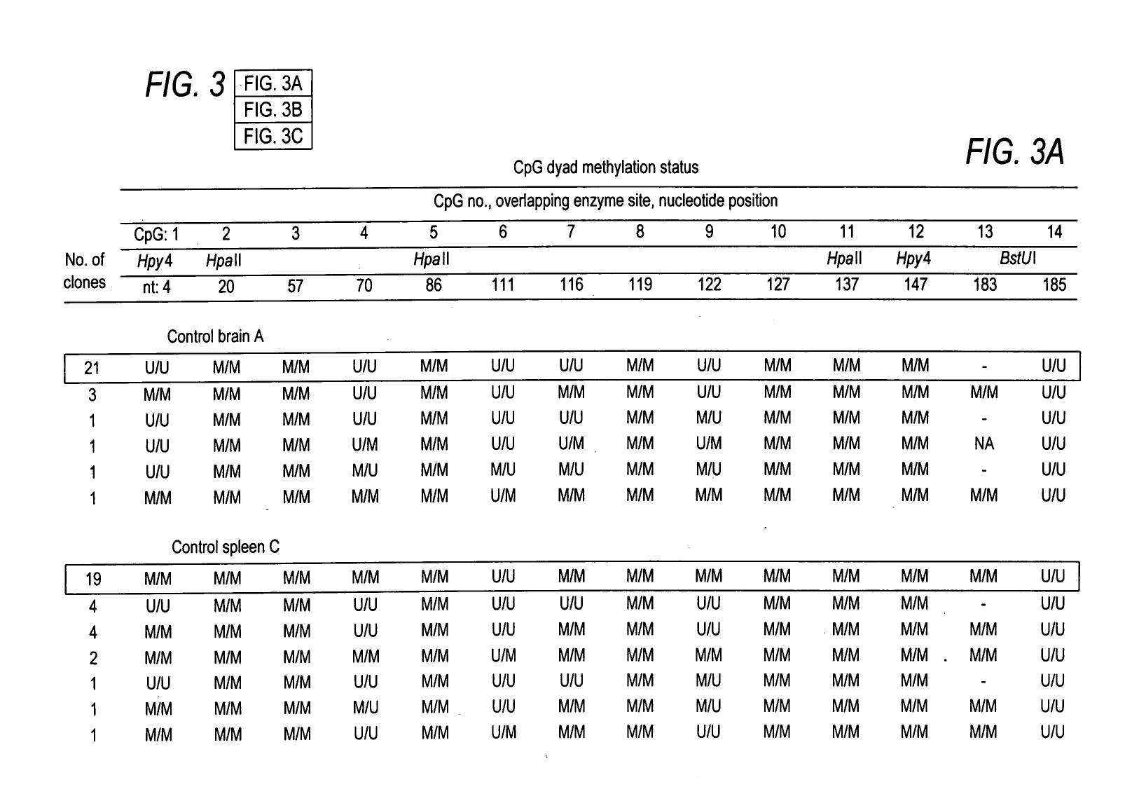

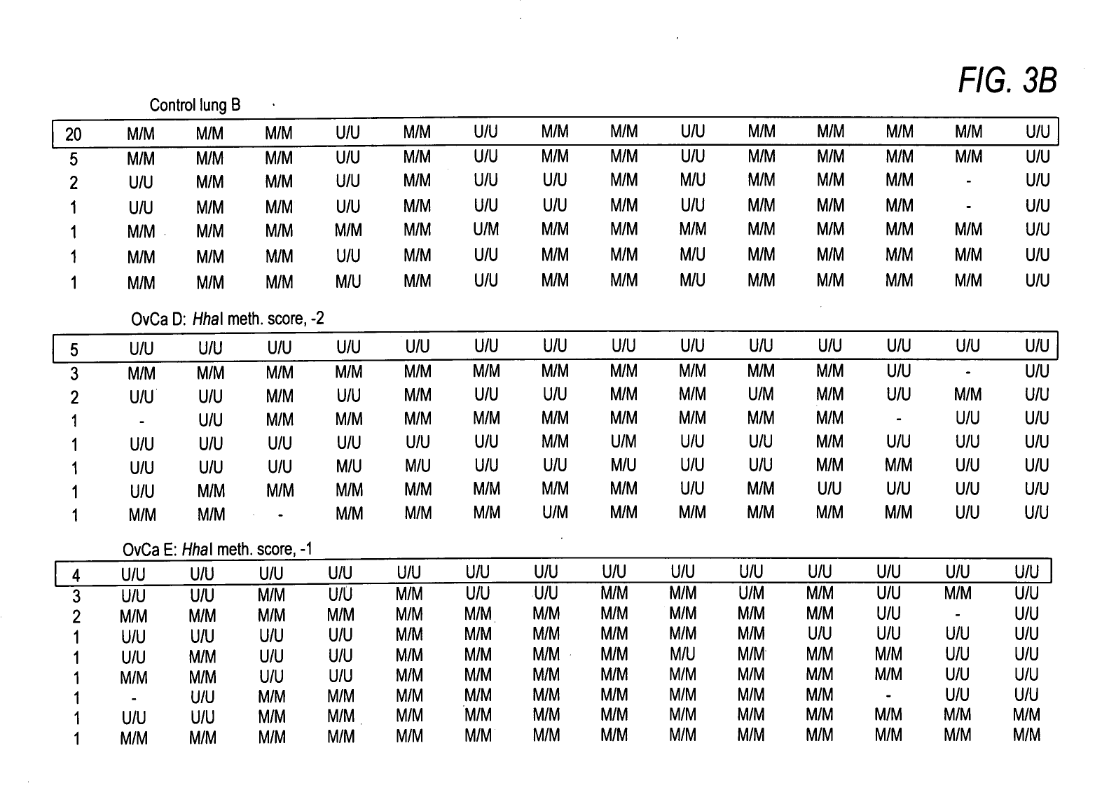

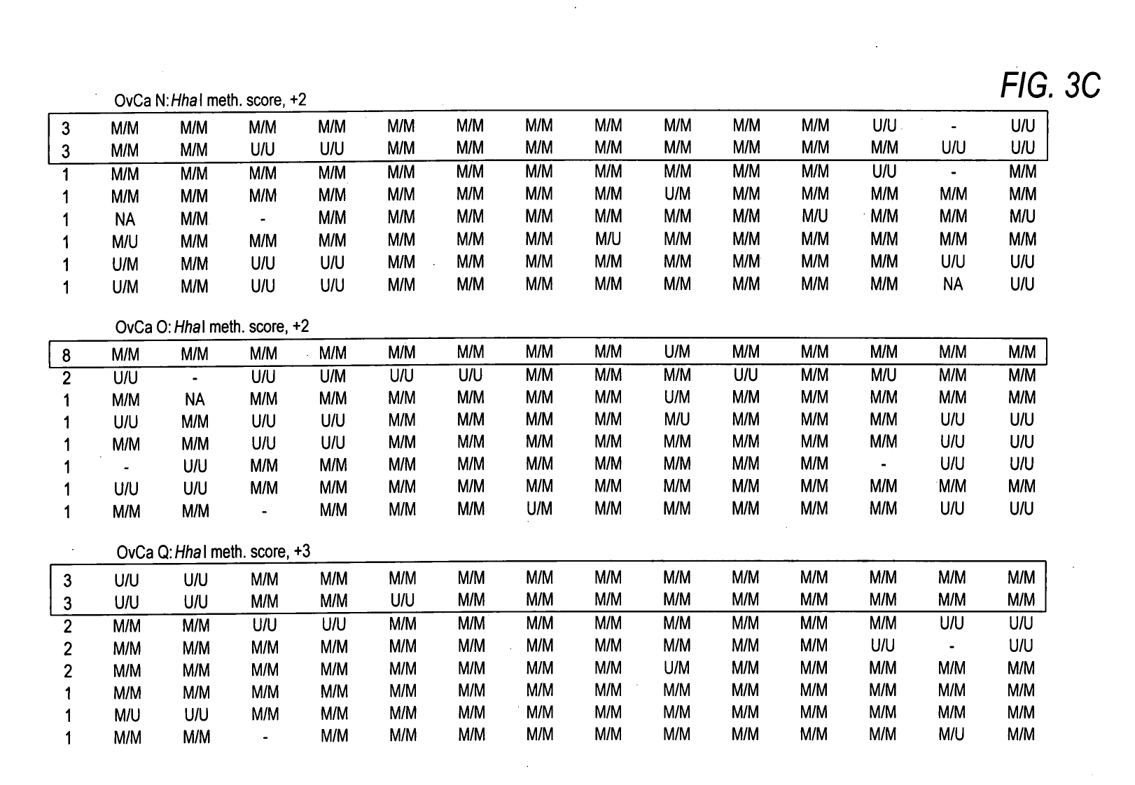

As used herein, “normal” refers to an individual who has not shown any cancer symptoms or has not been diagnosed with cancer. “Normal,” “reference,” and “reference sample,” according to the invention, refer to a sample taken from normal individuals. A normal tissue sample, for example, refers to the whole or a piece of a tissue isolated from, for example, rectum, breast, prostate, ovary, brain, kidney, blood, lung, colon, pancreas or bladder tissue post-mortem from an individual who was not diagnosed with cancer and whose corpse does not show any symptoms of cancer at the time of tissue removal. In an embodiment, the reference sample does not have to be derived from the same type of tissue in which a test sample is compared to. In an embodiment, the reference sample does not have to be derived from the same subject in which a test sample is compared to. In an embodiment, the normal tissue is ovarian epithelial cells or embryonic kidney remnant. The methylation status of a normal reference, or a reference sample, is shown in FIGS. 3, 5A and 7E.

As used herein, the term “sample” means any bodily secretions, biological fluid, cell, tissue, organ or portion thereof, that contains genomic DNA suitable for methylation detection via the methods. A test sample can include or be suspected to include a neoplastic cell, such as a cell from the cheek, rectum, breast, prostate, ovary, blood, brain, kidney, lung, colon, pancreas or bladder tissue that contains or is suspected to contain a neoplastic cell. The term includes samples present in an individual as well as samples obtained or derived from the individual. For example, a sample can be a histological section of a specimen obtained by biopsy, or cells that are placed in or adapted to tissue culture. A sample further can be a subcellular fraction or extract, or a crude or substantially pure nucleic acid molecule or protein preparation. A reference sample can be used to establish a reference methylation status or methylation pattern and, accordingly, can be derived from the source tissue that has the particular phenotypic characteristics to which the test sample is to be compared.

As used herein, the term “disease-free survival” refers to the lack of tumor recurrence and/or spread and the fate of a patient after diagnosis, for example, a patient who is alive without tumor recurrence.

As used herein, the term “overall survival” refers to the fate of the patient after diagnosis, regardless of whether the patient has a recurrence of the tumor. As used herein, the term “risk of recurrence” refers to the probability of tumor recurrence or spread in a patient subsequent to treatment of cancer. Tumor recurrence refers to further growth of neoplastic or cancerous cells after treatment of cancer. Particularly, recurrence can occur when further cancerous cell growth occurs in the cancerous tissue. Tumor spread refers to dissemination of cancer cells into local or distant tissues and organs, for example during tumor metastasis. Tumor recurrence, in particular, metastasis, is a significant cause of mortality among patients who have undergone surgical treatment for cancer.

As used herein, the term “in combination” refers to the use of more than one therapies (e.g., prophylactic and/or therapeutic agents). The use of the term “in combination” does not restrict the order in which therapies (e.g., prophylactic and/or therapeutic agents) are administered to a subject with cancer.

As used herein, the terms “subject” and “patient” are used interchangeably. As used herein, a subject is preferably a mammal such as a non-primate (e.g., cows, pigs, horses, cats, dogs, rats etc.) and a primate (e.g., monkey and human), most preferably a human. In a specific embodiment, the subject is a non-human animal. In another embodiment, the subject is a farm animal (e.g., a horse, a pig, a lamb or a cow) or a pet (e.g., a dog, a cat, a rabbit or a bird). In another embodiment, the subject is an animal other than a laboratory animal or animal model (e.g., a mouse, a rat, a guinea pig or a monkey). In a preferred embodiment, the subject is a human.

As used herein, the term “microarray” refers broadly to both “DNA microarrays” and “DNA chip(s)”, as recognized in the art, which encompasses all art-recognized solid supports, and encompasses all methods for affixing nucleic acid molecules thereto or synthesis of nucleic acids thereon. In a specific embodiment, the microarray utilizes a high throughput method.

### 5. DETAILED DESCRIPTION OF INVENTION

The inventors of the present application have discovered that relative to normal (non-cancerous) somatic tissues, cancers can display both hypomethylation and hypermethylation within one or more specific subregions within a genomic DNA sequence. Accordingly, the present invention is directed to a method for diagnosing cancer based on DNA methylation differences at specific genomic CpG dinucleotides. The present method also provides for a hairpin bisulfite PCR for determining strand-specific methylation status at genomic CpG dinucleotides.

Evidence had been provided for cross-talk between demethylation and de novo methylation pathways in tumorigenesis (Pogribny, et al. (1997). Cancer Lett., 115, 31-38) and in Arabidopsis containing an antisense DNA methyltransferase transgene (Jacobsen, et al. (1997). Science, 277, 1100-1103). However, hypermethylation of 5′ regions of tumor suppressor genes and hypomethylation of LINE1 interspersed repeats, satellite DNA, and promoter regions of cancer-testes antigen genes (Santourlidis, et al. (1999). Prostate, 39, 166-174; Ehrlich, et al. (2002). Oncogene, 21, 6694-6702, 2002; Kaneda, et al. (2004). Cancer Sci., 95, 58-64; Ehrlich et al. unpublished data) are statistically independent of each other, even though all such changes are linked to cancer. Although hypermethylation at certain DNA sequences and hypomethylation at others in cancer are not associated with one another, the present invention shows that both cancer-linked hypo- and hypermethylation are targeted to NBL2 repeats. Not to be bound by any theory, a chromatin structure change in NBL2 arrays occurs during oncogenesis which may predispose the sequence to both demethylation and de novo methylation in cis. Alternatively, NBL2 arrays, which have a high overall m⁵CpG content, might first be demethylated during tumorigenesis and the resulting chromatin structure change might favor further demethylation as well as de novo methylation.

Previous bisulfite-based genomic sequencing studies of cancer DNA, usually involving unmethylated CpG-rich promoters that become hypermethylated, indicate mostly homogeneous increases in methylation of CpG's within a small region (Melki, et al. (1999). Cancer Res., 59, 3730-3740; Rush, et al. (2004). Cancer Res., 64, 2424-2433; Amoreira, et al. (2003). Nucleic Acids Res., 31, 75-77). There may be several reasons for NBL2 displaying surprisingly complex, non-random patterns of methylation change during carcinogenesis. It is apparently not a gene, and its methylation status probably confers no selective advantage to a developing tumor. This is unlike the situation with promoters of tumor suppressor genes whose almost complete methylation can benefit the growing tumor by repressing transcription or stabilizing this repression. Also, unlike most DNA regions from cancers analyzed by genomic sequencing, NBL2 normally has very low levels of methylation at some CpG's and complete methylation at many others so that both cancer-linked increases and decreases of DNA methylation can be observed. Furthermore, it seems to be an unusually frequent target for multiple methylation changes during carcinogenesis. As such, it is a good candidate for a cancer marker as well as a source of insight into cancer-linked epigenetic alterations without the skewing of DNA methylation patterns by oncogenic selection pressures.

Specifically, the inventors discovered that one or more specific subregions within the NBL2, a tandem 1.4-kb DNA repeat, exhibits variation in methylation status at genomic CpG dinucleotide sequences of ovarian carcinomas and Wilms tumors as compared to normal somatic tissues. This primate-specific sequence (Thoraval, et al. (1996). Genes Chromosomes Cancer, 17, 234-244) is CpG-rich (61% C+G; 5.7% CpG). It is present in about 200-400 copies per haploid human genome, mostly in the vicinity of the centromeres of four of the five acrocentric chromosomes (Nishiyama, et al. (2005). Cancer Biol. Ther., 4, 440-448), repeat-rich regions for which only little sequence information is available.

Although not intending to be bound by any mechanism of action, the inventors discovered that methylation in a subregion (about 0.2 kb) of NBL2 from diverse normal somatic tissues displayed symmetrical methylation at seven CpG positions and no methylation or only hemimethylation at two others. Unexpectedly, 56% of cancer DNA clones from diverse types of cancer, such as ovarian carcinomas and Wilms tumors, had decreased methylation at some of the seven CpG sites as well as increased methylation at one or both of the two other CpG sites. All 146 DNA clones from ten cancer samples could be distinguished from all 91 somatic control clones by assessing methylation changes at three of these CpG sites. The inventors also discovered in the present invention that combined Southern blot and genomic sequencing data indicate that some of the cancer-linked alterations in CpG methylation are occurring with considerable sequence specificity, despite the finding that NBL2 does not seem to be a gene. The present invention relates to use of NBL2 as an epigenetic cancer marker and for elucidating the nature of epigenetic changes in cancer. Accordingly, the present invention relates to diagnostic or prognostic assays for cancer based on analysis of altered methylation status at specific CpG dinucleotide sequences within subregions of the genomic target NBL2. Furthermore, the present invention also provides specific diagnostic nucleotide positions that exhibit variations in CpG methylation status in a disease state compared to a normal state, and, thus, are useful for practicing the methods of the present invention.

5.1 Diagnosis and Prognosis of Cancer Using NBL2 as a Marker

The present invention provides diagnostic and prognostic methods for cancers that are characterized by change in methylation status of genomic CpG dinucleotide sequences in subregions within the NBL2 genomic sequence. Also provided are specific markers and corresponding nucleic acid molecules in one or more subregions of NBL2 that are useful for the detection of a change in methylation status of genomic CpG dinucleotide sequences that can be correlated to the presence of or susceptibility to cancer in an individual. This invention is also directed to methods for predicting the susceptibility of an individual to cancer that is characterized by a change in methylation status of genomic CpG dinucleotide sequences in at least one subregion of NBL2 as compared with the methylation status of the genomic CpG dinucleotide sequences in that subregion of NBL2 exhibited in the absence of the condition.

In various distinct embodiments, the present invention is based, in part, on the identification of reliable CpG dinucleotide sequences as markers in at least one subregion of the NBL2 sequence for the improved prediction of susceptibility, diagnosis and staging of cancer. The invention provides reliable genomic sequences in one or more subregions of the NBL2 sequence for use in the diagnostic and prognostic methods provided by the present invention.

In a preferred embodiment, NBL2 has a nucleotide sequence of GenBank Accession Nos. Y10752, U59100 or AC0128692. In the most preferred embodiment, NBL2 has a nucleotide sequence of SEQ ID NO:2. In other preferred embodiments, other NBL2 nucleotide sequences that are useful in the present invention includes nucleic acid molecules that are at least 80% identical to SEQ ID NO:2 or hybridize to the complement of SEQ ID NO:2. In the most preferred embodiment, the subregion within NBL2 used in the method of the present invention has a nucleotide sequence of SEQ ID NO:1 or SEQ ID NO:8. In other embodiments, the subregion within NBL2 is at least 80% identical to SEQ ID NO:1 or 8.

The invention provides methods of detecting and diagnosing cancer in a subject by identifying a change in methylation status in one or more genomic CpG dinucleotide sequences of NBL2. In specific embodiments, the one or more genomic CpG dinucleotide sequence is within a subregion of the NBL2. In a specific embodiment, the subregion is about 100, 200, 300, 400, or 500 b.p.

In a most preferred embodiment, methylation status is determined in a 0.2-kb subregion of NBL2 in ovarian carcinomas, Wilms tumors, and diverse control tissues by hairpin-bisulfite genomic sequencing, which detects every 5-methylcytosine on covalently linked, complementary strands. Blot hybridization of 33 cancer DNAs digested with CpG methylation-sensitive enzymes confirmed that NBL2 arrays are unusually susceptible to cancer-linked hypermethylation and hypomethylation, consistent with our novel genomic sequencing findings.

In one embodiment, the invention provides a method for identification of a change in methylation status in one or more genomic CpG dinucleotide sequences associated with cancer in an individual by obtaining a biological sample comprising genomic DNA from the individual; measuring the methylated status of one or more genomic CpG dinucleotide sequences of the genomic NBL2 sequence in the sample, and comparing the methylation status of one or more genomic CpG dinucleotide sequences in the sample to a reference methylated status of one or more genomic CpG dinucleotide sequences, wherein a difference in the methylation status of one or more genomic CpG dinucleotide sequences in the sample compared to the reference identifies an association of the individual with cancer.

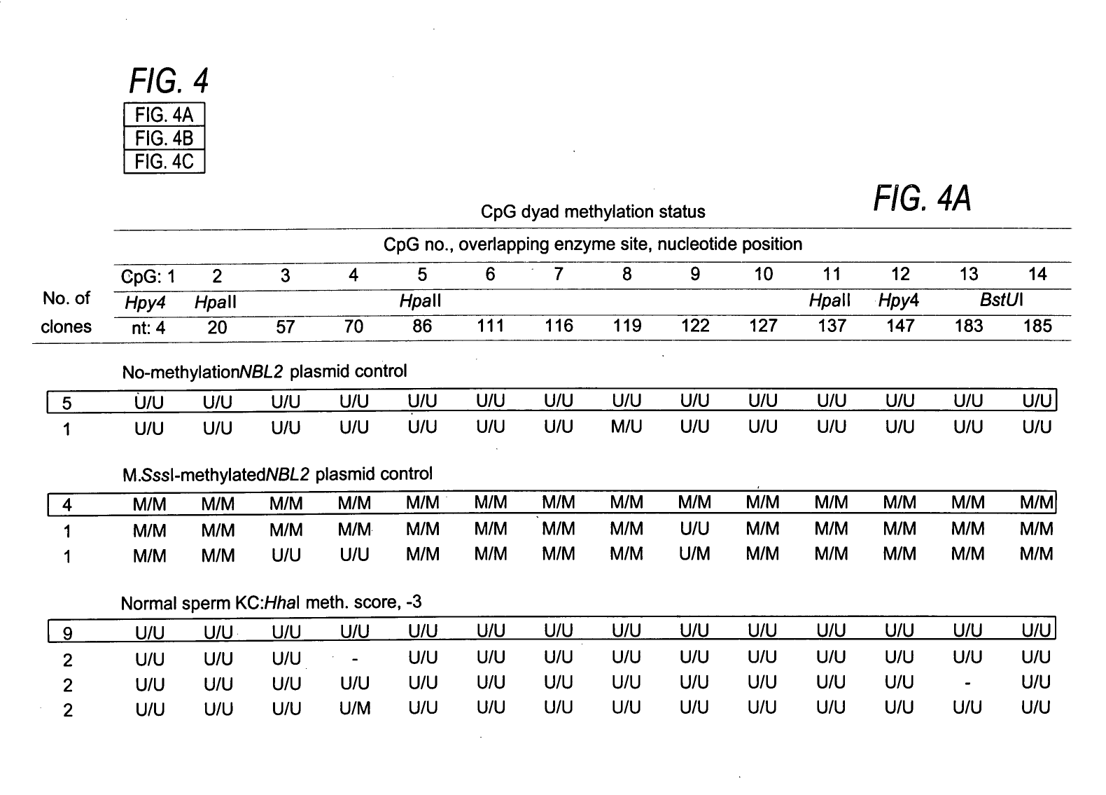

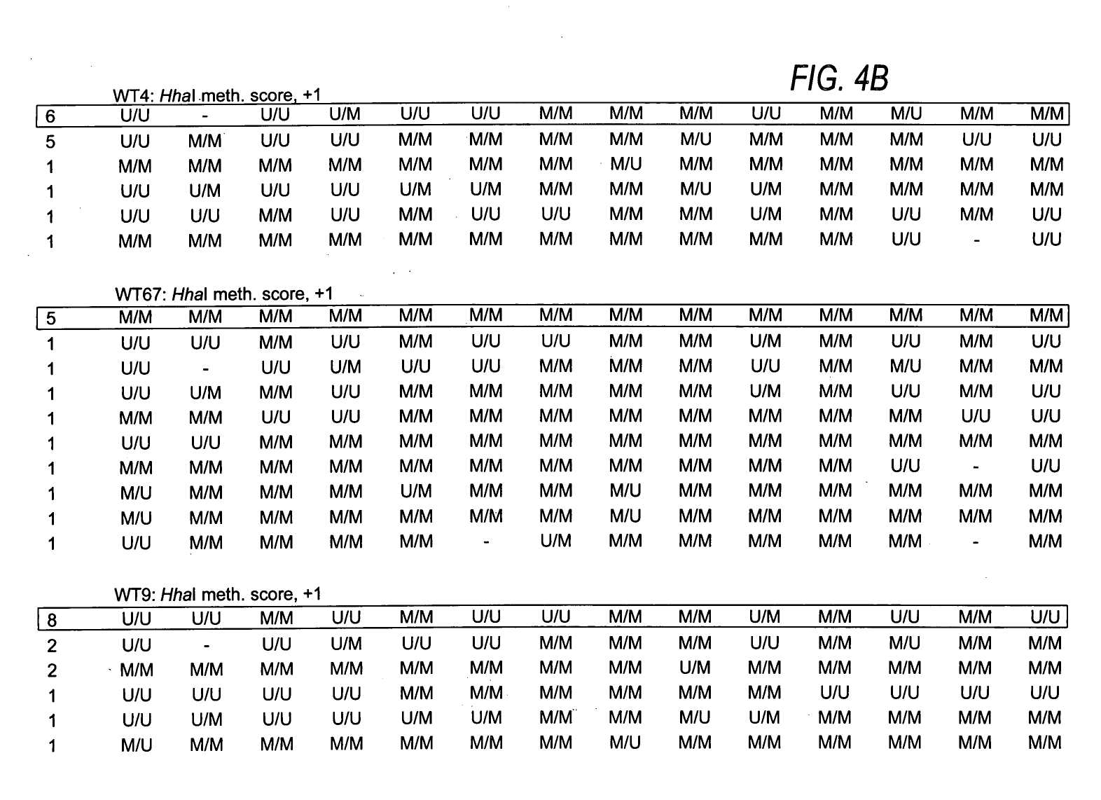

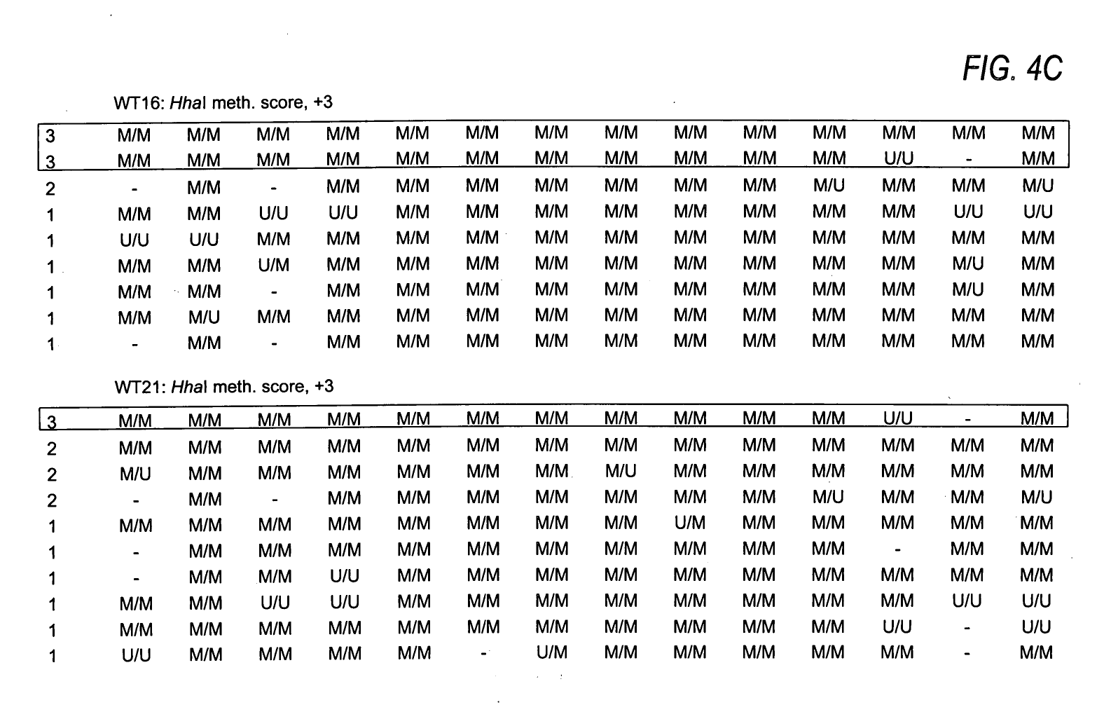

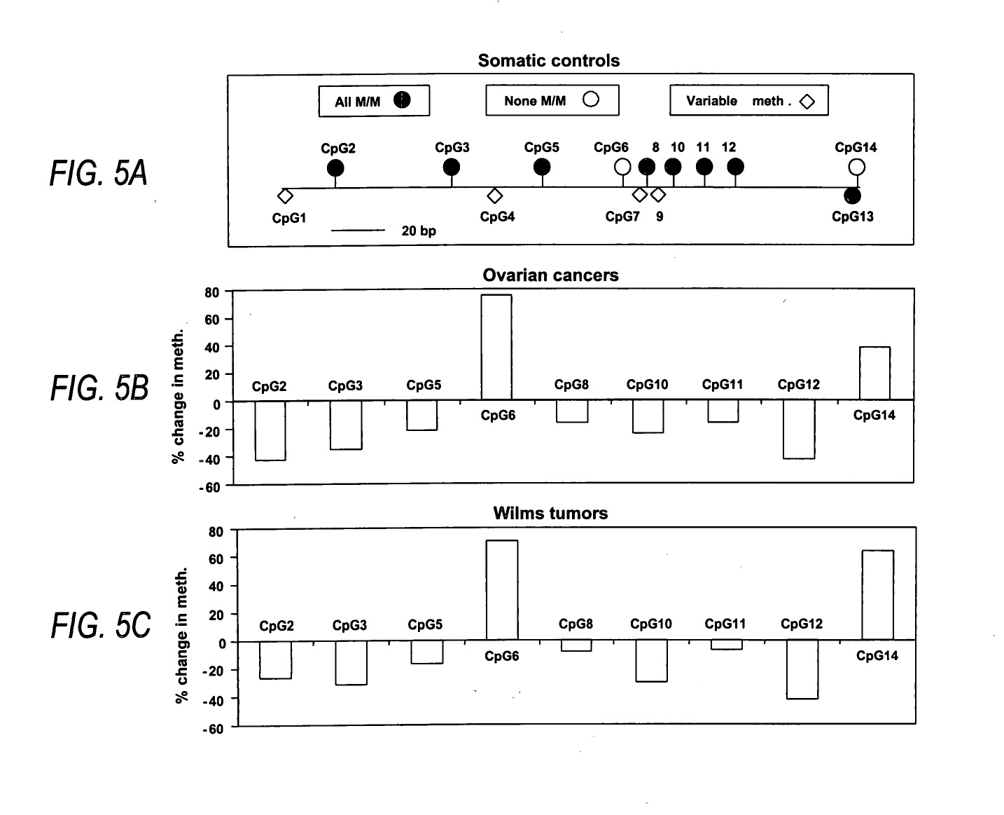

The present inventors have discovered both hypomethylation and hypermethylation within the same molecular clones from cancers. First, CpG sites were identified with invariant methylation status in somatic control tissues (brain, spleen, and lung from different normal individuals). There was a surprisingly high degree of conservation of a complex methylation pattern at NBL2 in the normal somatic tissues (FIG. 3 and FIG. 5A) in contrast to the usual findings of either very heterogeneous methylation patterns from molecule to molecule or almost complete methylation or lack of methylation in a given DNA region (Melki, et al. (1999). Cancer Res., 59, 3730-3740; Millar, et al. (2000). J. Biol. Chem., 275, 24893-24899; Amoreira, et al. (2003). Nucleic Acids Res., 31, 75-77). Among the 91 NBL2 DNA clones from somatic controls subject to hairpin-bisulfite PCR, 7 of the 14 CpG sites were always symmetrically methylated (CpG2, 3, 5, 8, 10, 11, and 12). Two nonadjacent CpG's were never symmetrically methylated (CpG6 and 14). One of these, CpG14, was always U/U, and the other, CpG6, was usually U/U but occasionally U/M or M/U. CpG13, which is exactly adjacent to always-unmethylated CpG14 was often replaced by GpG, and hence could not be methylated. However, whenever it was not replaced, it was always M/M despite its immediate U/U neighbor (FIGS. 3 and 5A). Normal sperm showed a complete absence of symmetrical CpG methylation in the examined NBL2 subregion (FIG. 4), consistent with previous results from various tandem DNA repeats (Ehrlich, 2002). Oncogene, 21, 5400-5413.

None of the 146 cancer DNA clones had the conserved methylation pattern of normal somatic controls (FIG. 3 and FIG. 4). Moreover, 56% of the cancer clones had a mixture of both hypomethylated and hypermethylated CpG sites. These methylation changes were defined by the loss of the normally conserved M/M status at CpG2, 3, 5, 8, 10, 11, or 12 and the gain of M/M status at CpG6 or 14, sites never normally symmetrically methylated (FIGS. 3, 4, and 5). The overall methylation status at each of these 9 CpG sites in the cancers was significantly different from that in the somatic controls (p<0.005; p-value adjusted for multiple comparisons).

The inventors have discovered CpG sites with preferred methylation changes in cancers. Some normally M/M CpG sites (FIG. 5A) appeared to be more likely to become demethylated in both the ovarian carcinomas and Wilms tumors than others (FIGS. 5B and C). To test the significance of this finding, a pairwise comparison of methylation changes in cancer clones at the seven normally M/M sites and also at the two normally unmethylated CpG dyads was performed. In both the Wilms tumor group and the ovarian carcinoma group, the following significant differences were observed: demethylation at CpG12 was more frequent than at CpG8 or 11, demethylation at CpG2 was more frequent than at CpG5; and demethylation at CpG3 was more frequent than at CpG11 (p<0.05 after adjustment for multiple comparisons). With respect to the two positions that were never normally symmetrically methylated, CpG6 was significantly more prone to cancer-associated hypermethylation (conversion to M/M) than CpG14 (p<0.00001) in ovarian carcinomas, although not in the Wilms tumors.

There is also evidence of cancer-linked epigenetic patterning involving multiple CpG positions in the sequenced NBL2 region. Eleven cancer clones derived from four cancers had the following methylation status: CpG4, U/M; CpG12, M/U; CpG1, 3, 5, 6, and 10, U/U; CpG7, 8, 9, 11, 13, and 14, M/M (FIGS. 3 and 4: first row for WT4, second row for WT9 and OvCaO, and third row for WT67). This methylation pattern constitutes changes from the normally conserved methylation status of the five underlined CpG sites.

The inventors have discovered that cancer and somatic control clones can be distinguished by methylation status at several CpG's. A few CpG sites whose methylation status could be used to distinguish all the cancer-derived molecular clones from all the somatic control clones were tested. Such sites have 100% predictive power by generating a classification tree from the data. All but two of the cancer-derived clones displayed symmetrical methylation at CpG6 (M/M) or demethylation at CpG10 (U/U or U/M); none of the control clones had these epigenetic attributes. The two exceptional tumor clones could not display this hypomethylation because CpC or CpT replaced CpG6. Those two clones (last row in WT67 and 21, FIG. 4) exhibited hypermethylation at CpG14, which distinguishes them from all control clones. Therefore, all cancer clones were different from all somatic control clones by hypomethylation at CpG10 or hypermethylation at CpG6 or CpG14. The ability to distinguish all NBL2 cancer clones from all NBL2 somatic control clones also demonstrates the purity of the cancer DNA samples used for this analysis.

In a preferred embodiment, NBL2 has a nucleotide sequence set forth in SEQ ID NO: 2 or a nucleotide sequence that shares at least 80% sequence identity with SEQ ID NO:2. In a preferred embodiment, the nucleotide sequence shares at least 90-95% sequence identity with SEQ ID NO:2. In additional embodiments, the methylation status of genomic CpG dinucleotide sequences is measured for one, two, three, four, five, six, seven, eight, nine, ten, eleven, twelve, thirteen, fourteen, fifteen, sixteen, seventeen, eighteen, or more, CpG dinucleotide sequences in a subregion of the genomic marker sequence NBL2. Nucleic acids that are portions of (preferably at least 15, 20 nucleotide portions) subregion of the genomic marker sequence NBL2 are also provided as probes or primers in the present invention. In a specific embodiment, the subregion has a nucleotide sequence set forth in SEQ ID NO:1 or 8. In a specific embodiment, the one or more genomic CpG dinucleotide sequences in subregion 1 are CpG2, CpG3, CpG5, CpG6, CpG8, CpG10, CpG11, CpG12, CpG13, and CpG14. In a specific embodiment, the one or more genomic CpG dinucleotide sequences in subregion 2 are CpG21, CpG22, CpG23, CpG24, CpG25, CpG26, CpG27, CpG28, CpG29, CpG30, CpG31, CpG32, CpG33, CpG34, CpG35 CpG36, CpG37. In specific embodiments, the subregion of the NBL2 sequence retains certain CpG dinucleotide sequences that are useful for the diagnosis and prognosis methods of the present invention. In specific embodiments, the change in methylation status for the CpG dinucleotide sequence has at least 60%, 70%, 80%, 90%, or 95% predictive power for cancer.

In addition to detecting the status of methylation of the genomic CpG dinucleotide sequences within a subregion of the genomic NBL2 sequence, the present invention also allows for the detection of patterns of methylation. The methylation status of two or more dinucleotide sequences provides a specific pattern of methylation. Accordingly, the invention provides a method for identification of a change in methylation pattern in two or more genomic CpG dinucleotide sequences associated with cancer in an individual by obtaining a biological sample comprising genomic DNA from the individual; measuring the methylated status of two or more genomic CpG dinucleotide sequences of the genomic NBL2 sequence in the sample, and comparing the methylation pattern of two or more genomic CpG dinucleotide sequences in the sample to a reference methylated status of two or more genomic CpG dinucleotide sequences, wherein a difference in the methylation pattern of two or more genomic CpG dinucleotide sequences in the sample compared to the reference identifies an association of the individual with cancer.

The methylation status and the patterns of methylation of the genomic CpG dinucleotide sequences can provide a variety of information about the cancer and can be used, for example, to diagnose or predict susceptibility for a particular type, class or origin of cancer; to diagnose the presence of cancer in the individual; to predict the course of the cancer in the individual; to predict the susceptibility to cancer in the individual, to stage the progression of the cancer in the individual; to predict the likelihood of disease-free survival for the individual; to predict the likelihood of overall survival for the individual; to predict the likelihood of recurrence of cancer for the individual; to determine the effectiveness of a treatment course undergone by the individual.

Also provided are nucleic acid probes, linker and primer sequences derived from the genomic NBL2 sequence which are useful for detection of genomic CpG dinucleotide sequences that exhibit methylation changes associated with cancer.

The prognostic methods of the invention are useful for determining if a patient is at risk for recurrence. Cancer recurrence is a concern relating to a variety of cancers. One explanation for cancer recurrence is that patients with relatively early stage disease, for example, stage II or stage III, already have small amounts of cancer spread outside the affected organ that were not removed by surgery. These cancer cells, referred to as micrometastases, cannot typically be detected with currently available tests.

The prognostic methods of the invention can be used to identify surgically treated patients likely to experience cancer recurrence so that they can be offered additional therapeutic options, including preoperative or postoperative adjuncts such as chemotherapy, radiation, biological modifiers and other suitable therapies. The methods are especially effective for determining the risk of metastasis in patients who demonstrate no measurable metastasis at the time of examination or surgery.

The prognostic methods of the invention also are useful for determining a proper course of treatment for a patient having cancer. A course of treatment refers to the therapeutic measures taken for a patient after diagnosis or after treatment for cancer. For example, a determination of the likelihood for cancer recurrence, spread, or patient survival, can assist in determining whether a more conservative or more radical approach to therapy should be taken, or whether treatment modalities should be combined. For example, when cancer recurrence is likely, it can be advantageous to precede or follow surgical treatment with chemotherapy, radiation, immunotherapy, biological therapy, gene therapy, vaccines, and the like, or adjust the span of time during which the patient is treated.

This invention provides methods for determining a prognosis for survival for a cancer patient. In an embodiment, the method comprises (a) determining the methylation status of one or more CpG dinucleotide sequence in a subregion of NBL2 in a neoplastic cell-containing sample from the cancer patient, and (b) comparing the methylation status in the sample to a reference methylation status, wherein a change in methylation status of one or more CpG dinucleotide sequence in a subregion of NBL2 in the sample correlates with decreased survival of the patient.

This invention also provides a method for monitoring the effectiveness of a course of treatment for a patient with cancer. The method comprises (a) determining the methylation status of one or more CpG dinucleotide sequence in a subregion of NBL2 in a neoplastic cell-containing sample from the cancer patient, and (b) comparing the methylation status in the sample to a reference methylation status, wherein an unchange in methylation status of one or more CpG dinucleotide sequence in a subregion of NBL2 in the sample indicates the effectiveness of the treatment.

It is understood that a reference methylation status has to correspond to one or more genomic CpG dinucleotide sequences present in a corresponding sample that allows comparison to the desired phenotype. For example, in a diagnostic application, a reference methylation status can be based on a reference sample or a normal sample that is derived from a cancer-free origin so as to allow comparison to the biological test sample for purposes of diagnosis. In a method of staging a cancer, it can be useful to apply in parallel a series of reference methylation status, each based on a sample that is derived from a cancer that has been classified based on parameters established in the art, for example, phenotypic or cytological characteristics, as representing a particular cancer stage so as to allow comparison to the biological test sample for purposes of staging. In addition, progression of the course of a condition can be determined by determining the rate of change in the methylation status (when one CpG dinucleotide sequence is involved) or the pattern of methylation (when two or more CpG dinucleotide sequences are involved) of genomic CpG dinucleotide sequences by comparison to reference methylation status or pattern of methylation derived from reference samples that represent time points within an established progression rate. It is understood, that the user will be able to select the reference sample and establish the reference methylation status or methylation pattern based on the particular purpose of the comparison.

The methods of the invention can be applied to the characterization, classification, differentiation, grading, staging, diagnosis, or prognosis of a condition characterized by a change in methylation status of one or more genomic CpG dinucleotide sequences or a change in methylation pattern of two or more genomic CpG dinucleotide sequences that is distinct from the methylation status or methylation pattern of genomic CpG dinucleotide sequences exhibited in the absence of cancer.

The present invention is directed to the use of methylation status or methylation pattern of CpG dinucleotide sequences in a subregion of NBL2 to classify and predict different kinds of cancer, or the same type of cancer in different stages. The present invention also provides a useful tool for cancer diagnosis, or preferably, for detection of premalignant changes. When combined with the development of sensitive, non-invasive disease diagnosis (e.g. a blood test, blood pressure, cancer staging, age, life style, family history, disease history, molecular biological parameters, cellular parameters, histological parameters, physiological parameters, anatomical parameters, pathological parameters, and gene expression) this may provide a viable method to screen subjects at risk for cancer as well as to monitor cancer progression and response to treatment.

5.2 Methods for Determining the Methylation Status of a Genomic Sequence

Methylation of CpG dinucleotide sequences can be measured using any of a variety of techniques used in the art for the analysis of specific CpG dinucleotide methylation status. Methylation of CpG dinucleotide sequences can be measured by employing cytosine conversion based technologies, which rely on methylation status-dependent chemical modification of CpG sequences within isolated genomic DNA, or fragments thereof, followed by DNA sequence analysis. Chemical reagents that are able to distinguish between methylated and non-methylated CpG dinucleotide sequences include hydrazine, which cleaves the nucleic acid, and bisulfite treatment. Bisulfite treatment followed by alkaline hydrolysis specifically converts non-methylated cytosine to uracil, leaving 5-methylcytosine unmodified as described by Olek (1996). Nucleic Acids Res., 24, 5064-6, or Frommer, et al. (1992). Proc. Natl. Acad. Sci. USA, 89, 1827-1831. The bisulfite-treated DNA can subsequently be analyzed by conventional molecular techniques, such as PCR amplification, sequencing, and detection comprising oligonucleotide hybridization.

In the most preferred embodiment, the invention provides a robust and ultra high-throughput technology further described in Section 5.2.1., for simultaneously measuring methylation at many specific sites in a genome. The invention further provides cost-effective methylation profiling of thousands of samples in a reproducible, well-controlled system. In particular, the invention allows implementation of a process, including sample preparation, bisulfite treatment, genotyping-based assay and PCR amplification that can be carried out on a robotic platform. In a specific preferred embodiment, the high-throughput method that is useful in the present invention incorporates pyro-sequencing for the de novo sequencing of a large genome in a large number of samples. Yang, et al., (2004), Nucleic Acids Res., 32(3)e38; Dupont, et al. (2004), Anal Biochem., 333(1), 119-27.

In a specific embodiment, the genomic DNA from a sample is treated with bisulfite and PCR is performed using PCR primers designed from the NBL2 sequence that are useful in the present invention to allow amplification of a pool of repeats. The sequence difference in this pool of amplified repeats can be quantitated by a number of means to determine the methylation status of the NBL2 subregions as discussed infra.

The change in methylation status can be measured in percentage change in methylation in a pool of amplified repeats. In specific embodiments, the % change in methylation is at least −60%, −50%, −40%, −30%, −20%, −10%, 10%, 20%, 30%, 40%, 50%, 60%, 70%, 90%, 99%, where a negative percentage indicates a change from methylation to unmethylation and a positive percentage indicates a change from unmethylation to methylation.

In a specific embodiment, the methylation status of specific genomic sequences in the DNA repeat NBL2 can be determined by hairpin-bisulfite PCR. Laird, et al. (2004). Proc. Natl. Acad. Sci. USA, 101, 204-209. This method is a new variant of the bisulfite-based genomic sequencing. In particular, bisulfite causes deamination of unmethylated C residues while methylated C residues are resistant to bisulfite (Frommer, et al. (1992). Proc. Natl. Acad. Sci. USA, 89, 1827-1831). Hairpin-bisulfite PCR allows analysis of methylation of every CpG (and C residue) in a given region on covalently linked DNA strands from a restriction fragment of interest. It also unambiguously differentiates naturally occurring sequence variation from bisulfite- and PCR-mediated C-to-T conversions at unmethylated cytosines. NBL2, a non-gene genomic sequence (Nishiyama, et al. (2005). Cancer Biol. Ther., 4, 440-448), is especially sensitive to multiple diverse DNA methylation changes during oncogenesis.

The following is a preferred embodiment of the invention which shows the use of the hairpin-bisulfite sequencing strategy and validation of the methylation status of the CpG dinucleotide sequences.

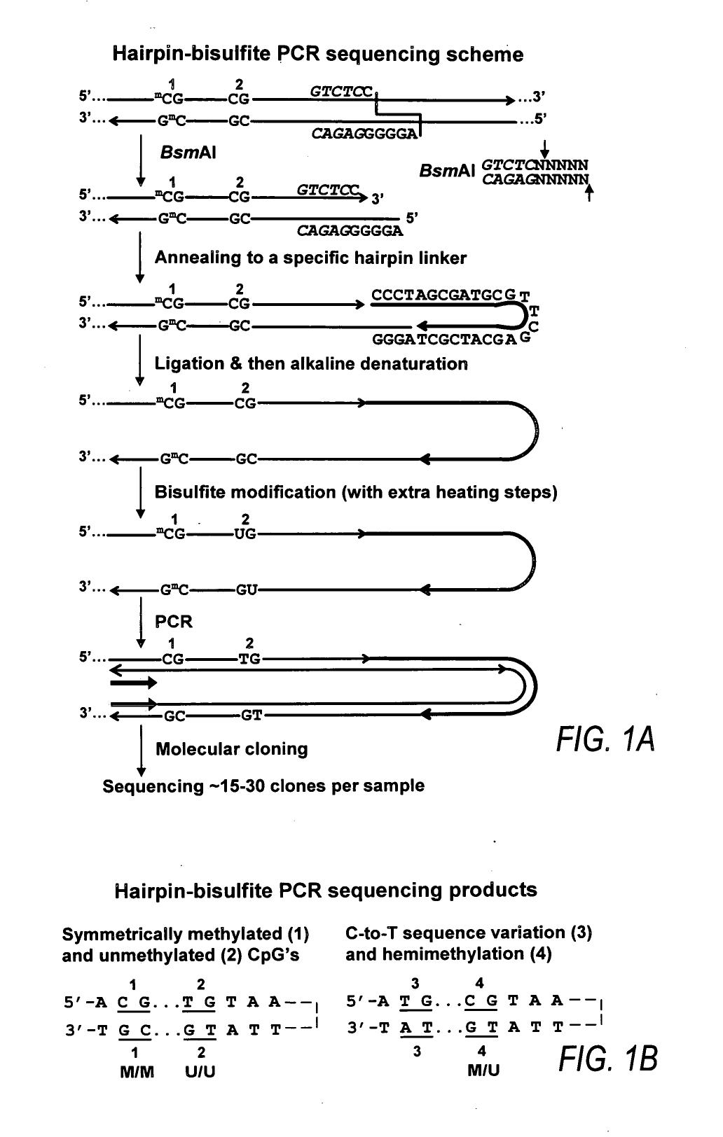

Hairpin-bisulfite PCR was performed using an NBL2 sequence (Y10752, GenBank) to design primers and the hairpin linker (Laird, et al. (2004). Proc. Natl. Acad. Sci. USA, 101, 204-209). FIG. 1 shows the outline of the hairpin-bisulfit PCR genomic sequencing methodology. Human DNA (0.5 μg) or NBL2-containing pDMHD-1 (50 ng) (Nagai, et al. (1999). Gene, 237, 15-20) plus 450 ng of λ DNA carrier were digested with 10 U of BsmAI and ligated to 5′CCCTAGCGATGCGTTCGAGCATCGCT-3′ (SEQ ID NO:3). The DNA was denatured with 0.6 M NaOH at 37° C. for 15 min followed by incubation in boiling water for 1 min. At hourly intervals during the 5-h bisulfite treatment, the sample was incubated 4 times in boiling water for 1 min. In an ultrafiltration device (Microcon-100; Millipore; Boyd & Zon, 2004), bisulfite-modified DNA was washed 3 times with water, desulfonated with 0.3 M of NaOH at 37° C. for 15 min, and eluted in 50 μl of 10 mM Tris-HCl, 1 mM EDTA, pH 7.5. The primers for subsequent PCR had a 3′ T or A corresponding to deamination products from a non-CpG C residue or its complement. The primers were F2-1,5′-TTTTTGTGGGTTTGTGTTAGT-3′(SEQ ID NO:5), and R2-2, 5′-CAAAAACATCTTTATTCCTCTA-3′(SEQ ID NO:6). F2-1 was replaced by F2-2,5′-AYGTGGTTTGGGTTAGGTAT-3′(SEQ ID NO:7), in the second round of PCR. Only the F2-2 primer had a CpG in the analogous unmodified genomic sequence (at positions 2 and 3). After denaturation at 94° C. for 15 min, PCR was performed (Hotstar, Qiagen) for 30 cycles on 2 μl of the bisulfite-treated DNA (94° C., 15 sec; 52° C., 15 sec, 72° C. 1 min, and a final extension at 72° C. for 5 min). Then, 1 μl of the product was amplified analogously for an additional 35 cycles. Purified fragments obtained by electrophoresis in a 1.5% agarose gel were used for cloning (TA Cloning Kit, Invitrogen), transformation (E. coli, Top10F), and sequencing (Translational Genomics Research Institute).

The 1.4-kb NBL2 repeat was analyzed by genomic sequencing using hairpin-bisulfite PCR (Laird et al., 2004. Proc. Natl. Acad. Sci USA, 101, 204-209). In DNA clones resulting from bisulfite treatment and PCR, a genomic m⁵CpG, the predominant site of vertebrate DNA methylation, will appear as CpG because it escaped bisulfite deamination, and an unmethylated CpG will become TpG due to cytosine deamination followed by amplification (FIG. 1A). In hairpin-bisulfite PCR, strand ligation results in the sequence information from both genomic strands of a DNA fragment being present in each strand of the resulting DNA clone (FIG. 1A). Corresponding CpG positions in the two halves of one strand of a DNA clone are compared to determine the methylation status of the template DNA molecule (FIG. 1B). For simplicity, the following terms are used for the DNA clones, which describe the CpG dyad methylation status of the molecule that gave rise to the clone: M/M, U/U, M/U, and U/M to describe CpG/CpG, TpG/TpG; CpG/TpG, and TpG/CpG, respectively, in the clone. Not only does hairpin-bisulfite PCR resolve a symmetrical methylation pattern at a CpG dyad from hemimethylation, but also it allows an unmethylated CpG to be unambiguously distinguished from germline C-to-T changes (FIG. 1B). This is especially useful for DNA repeats because of their appreciable sequence variation (Laird, et al. (2004). Proc. Natl. Acad. Sci. USA, 101, 204-209).

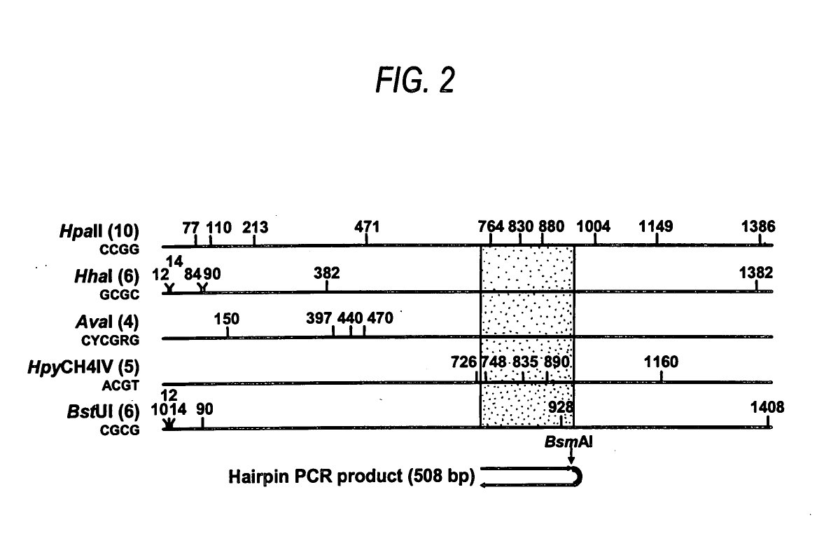

The portion of the NBL2 repeat that was amplified for this analysis is shown in gray in FIG. 2 along with restriction maps based upon a published 1.4-kb monomer. From the hairpin-bisulfite PCR on each normal tissue or cancer DNA, a single or predominant PCR band of the expected size (508 bp) was obtained from which 12 to 32 clones were generated and sequenced (FIG. 3 and FIG. 4). Given the originally self-complementary nature of the ligated DNA for bisulfite treatment and the specificity of bisulfite for denatured DNA, various controls were done to ensure that hairpin-bisulfite PCR did not yield artifacts. First, essentially only CpG methylation (Laird, et al. (2004). Proc. Natl. Acad. Sci. USA, 101, 204-209) was seen because postnatal tissues were examined (Dodge, et al. (2002). Gene, 289, 41-48). As expected, only 0-0.3% of non-CpG C residues per tissue sample were found (0.1% overall). Also, only 0.6% of C residues persisted as C in hairpin-bisulfite clones from an NBL2-containing E. coli plasmid (FIG. 4). In addition, the completeness of bisulfite modification was confirmed by digesting all hairpin-bisulfite PCR products with Tsp509I (recognizing 5′-AATT-3′). Gel electrophoresis of the digests indicated complete digestion due to bisulfite-mediated C deamination at genomic 5′-AACC-3′ or 5′-AACT-3′ in NBL2.

Also as expected, the two halves of each molecular clone, which are divided by the linker region, could be aligned by complementarity with only infrequent mismatches (NA sites in FIG. 3) other than those derived from bisulfite deamination of unmethylated C residues. Hairpin-bisulfite genomic sequencing of an M.SssI-methylated NBL2 plasmid showed that most of the CpG C residues were retained in the clones (FIG. 4). That 4% of CpG C residues in the M.SssI-methylated plasmid were converted to T residues probably reflects the common difficulty in driving CpG methylation by M.SssI to completion. In order to ensure that not only a few template molecules were amplified by using a 1:20 dilution of the bisulfite-treated DNA instead of the undiluted sample for PCR. A strong PCR product band was obtained with or without dilution from each sample. Thus, the sequenced molecular clones represent the heterogeneity in the sample DNA, which is consistent with their epigenetic and genetic sequence diversity (FIG. 3 and FIG. 4). Lastly, in an experiment with hairpin-bisulfite products amplified as 1:0, 1:3, 1:1, 3:1, and 0:1 mixtures of M.SssI-methylated and unmethylated NBL2 plasmid, the expected ratios of BstUI-sensitive sites (CGCG) to BstUI-resistant sites in the PCR product mixtures were obtained. Therefore, there was no appreciable selection during the PCR for templates that were methylated or unmethylated, as was sometimes found in PCR of bisulfite-treated DNA even when methylation-specific primers are avoided (Warnecke, et al. (1997). Nucleic Acids Res., 25, 4422-4426).

Other techniques for the analysis of bisulfite treated DNA can employ methylation-sensitive primers for the analysis of CpG methylation status with isolated genomic DNA as described by Herman, et al. (1996). Proc. Natl. Acad. Sci. USA, 93, 9821-9826, and in U.S. Pat. Nos. 5,786,146 and 6,265,171. Methylation sensitive PCR (MSP) allows for assessing the methylation status of virtually any methylated CpG position within, for example, the regulatory region of a gene, independent of the use of methylation-sensitive restriction enzymes. The DNA of interest is treated such that methylated and non-methylated cytosines are differentially modified, for example, by bisulfite treatment, converting all unmethylated, but not methylated cytosines to uracil, and subsequently amplified with primers specific for methylated versus unmethylated DNA and analyzed in a manner discernable by their hybridization behavior. PCR primers specific to each of the methylated and non-methylated states of the DNA are used in a PCR amplification. Products of the amplification reaction are then detected, allowing for the deduction of the methylation status of the CpG position within the genomic DNA. Other methods for the analysis of bisulfite treated DNA include methylation-sensitive single nucleotide primer extension (Ms-SNuPE) (Gonzalgo, et al. (1997). Nucleic Acids Res., 25, 2529-2531; and see U.S. Pat. No. 6,251,594), and the use of real time PCR based methods, such as the art-recognized fluorescence-based real-time PCR technique MethyLight™. (Eads, et al. (1999) Cancer Res., 59, 2302-2306, U.S. Pat. No. 6,331,393 to Laird, et al. (2004). Proc. Natl. Acad. Sci. USA, 101, 204-209; and see Heid, et al. (1996). Genome Res., 6, 986-994). It is understood that a variety of methylation assay methods can be used for the determination of the methylation status of particular genomic CpG positions. Methods which require bisulfite conversion include, for example, bisulfite sequencing, methylation-specific PCR, methylation-sensitive single nucleotide primer extension (Ms-SnuPE), MALDI mass spectrometry and methylation-specific oligonucleotide arrays and are described, for example, in U.S. patent application Ser. No. 10/309,803 and international application International Patent Application No.: PCT/US03/38582.

In another embodiment, methylation can be measured by employing a restriction enzyme based technology, which utilizes methylation sensitive restriction endonucleases for the differentiation between methylated and unmethylated cytosines. Restriction enzyme based technologies include, for example, restriction digest with methylation-sensitive restriction enzymes followed by Southern blot analysis, use of methylation-specific enzymes and PCR, restriction landmark genomic scanning (RLGS) and differential methylation hybridization (DMH).

Restriction enzymes characteristically hydrolyze DNA at and/or upon recognition of specific sequences or recognition motifs that are typically between 4- to 8-bases in length. Among such enzymes, methylation sensitive restriction enzymes are distinguished by the fact that they either cleave, or fail to cleave DNA according to the cytosine methylation state present in the recognition motif, in particular, of the CpG sequences. In methods employing such methylation sensitive restriction enzymes, the digested DNA fragments can be separated, for example, by gel electrophoresis, on the basis of size, and the methylation status of the sequence is thereby deduced, based on the presence or absence of particular fragments. Preferably, a post-digest PCR amplification step is added wherein a set of two oligonucleotide primers, one on each side of the methylation sensitive restriction site, is used to amplify the digested genomic DNA. PCR products are not detectable where digestion of the methylation sensitive restriction enzyme site occurs. Techniques for restriction enzyme based analysis of genomic methylation are well known in the art and include the following: differential methylation hybridization (DMH) (Huang, et al. (1999). Human Mol. Genet., 8, 459-70); Not I-based differential methylation hybridization (see e.g., WO 02/086163 A1); restriction landmark genomic scanning (RLGS) (Plass, et al. (1999). Genomics, 58, 254-62); methylation sensitive arbitrarily primed PCR (AP-PCR) (Gonzalgo, et al. (1997). Cancer Res., 57, 594-599); methylated CpG island amplification (MCA) (Toyota, et al. (1999). Cancer Res., 59, 2307-2312). Other useful methods for detecting genomic methylation are described, for example, in U.S. Pat. App. pub. No. 2003/0170684 or WO 04/05122.

Other methods can be used to screen for altered methylation patterns in genomic DNA, and to isolate specific sequences associated with these changes (Toyota, et al. (1999). Cancer Res., 59, 2307-12). Briefly, restriction enzymes with different sensitivities to cytosine methylation in their recognition sites are used to digest genomic DNAs from a sample prior to arbitrarily primed PCR amplification. Fragments that show differential methylation are cloned and sequenced after resolving the PCR products on high-resolution polyacrylamide gels. The cloned fragments are then used as probes for Southern analysis to confirm differential methylation of these regions.

5.2.1 DNA Array

In one embodiment, methylation status of genomic CpG dinucleotide sequences in a sample can be detected using an array of probes. In particular embodiments, a plurality of different probe molecules can be attached to a substrate or otherwise spatially distinguished in an array. Exemplary arrays that can be used in the invention include, without limitation, slide arrays, silicon wafer arrays, liquid arrays, bead-based arrays and others known in the art or set forth in further detail below. In preferred embodiments, the methods of the invention can be practiced with array technology that combines a miniaturized array platform, a high level of assay multiplexing, and scalable automation for sample handling and data processing.

An array of arrays, also referred to as a composite array, having a plurality of individual arrays that is configured to allow processing of multiple samples can be used. Exemplary composite arrays that can be used in the invention are described in U.S. Pat. No. 6,429,027 and U.S. 2002/0102578 and include, for example, one component systems in which each array is located in a well of a multi-well plate or two component systems in which a first component has several separate arrays configured to be dipped simultaneously into the wells of a second component. A substrate of a composite array can include a plurality of individual array locations, each having a plurality of probes and each physically separated from other assay locations on the same substrate such that a fluid contacting one array location is prevented from contacting another array location. Each array location can have a plurality of different probe molecules that are directly attached to the substrate or that are attached to the substrate via rigid particles in wells (also referred to herein as beads in wells).

In a particular embodiment, an array substrate can be fiber optical bundle or array of bundles, such as those generally described in U.S. Pat. Nos. 6,023,540, 6,200,737 and 6,327,410; and PCT publications WO9840726, WO9918434 and WO9850782. An optical fiber bundle or array of bundles can have probes attached directly to the fibers or via beads. Other substrates having probes attached to a substrate via beads are described, for example, in U.S. 2002/0102578. A substrate, such as a fiber or silicon chip, can be modified to form discrete sites or wells such that only a single bead is associated with the site or well. For example, when the substrate is a fiber optic bundle, wells can be made in a terminal or distal end of individual fibers by etching, with respect to the cladding, such that small wells or depressions are formed at one end of the fibers. Beads can be non-covalently associated in wells of a substrate or, if desired, wells can be chemically functionalized for covalent binding of beads. Other discrete sites can also be used for attachment of particles including, for example, patterns of adhesive or covalent linkers. Thus, an array substrate can have an array of particles each attached to a patterned surface.

In a particular embodiment, a surface of a substrate can include physical alterations to attach probes or produce array locations. For example, the surface of a substrate can be modified to contain chemically modified sites that are useful for attaching, either-covalently or non-covalently, probe molecules or particles having attached probe molecules. Chemically modified sites can include, but are not limited to the linkers and reactive groups set forth above. Alternatively, polymeric probes can be attached by sequential addition of monomeric units to synthesize the polymeric probes in situ. Probes can be attached using any of a variety of methods known in the art including, but not limited to, an ink-jet printing method as described, for example, in U.S. Pat. Nos. 5,981,733; 6,001,309; 6,221,653; 6,232,072 or 6,458,583; a spotting technique such as one described in U.S. Pat. No. 6,110,426; a photolithographic synthesis method such as one described in U.S. Pat. No. 6,379,895 or 5,856,101; or printing method utilizing a mask as described in U.S. Pat. No. 6,667,394. Accordingly, arrays described in the aforementioned references can be used in a method of the invention.

The size of an array used in the invention can vary depending on the probe composition and desired use of the array. Arrays containing from about 2 different probes to many millions can be made. Generally, an array can have from two to as many as a billion or more probes per square centimeter. Very high density arrays are useful in the invention including, for example, those having from about 10,000,000 probes/cm² to about 2,000,000,000 probes/cm² or from about 100,000,000 probes/cm² to about 1,000,000,000 probes/cm². High density arrays can also be used including, for example, those in the range from about 100,000 probes/cm² to about 10,000,000 probes/cm² or about 1,000,000 probes/cm² to about 5,000,000 probes/cm². Moderate density arrays useful in the invention can range from about 10,000 probes/cm² to about 100,000 probes/cm², or from about 20,000 probes/cm² to about 50,000 probes/cm². Low density arrays are generally less than 10,000 probes/cm² with from about 1,000 probes/cm² to about 5,000 probes/cm² being useful in particular embodiments. Very low density arrays having less than 1,000 probes/cm², from about 10 probes/cm² to about 1000 probes/cm², or from about 100 probes/cm² to about 500 probes/cm² are also useful in some applications.

The methods of the invention can be carried out at a level of multiplexing that is 96-plex or even higher including, for example, as high as 1,500-plex. An advantage of the invention is that the amount of genomic DNA used for detection of methylated sequences is low including, for example, less that 1 ng of genomic DNA. In one embodiment, the throughput of the methods can be 96 samples per run, with 1,000 to 1,500 methylation assays per sample (144,000 data points or more per run). In an embodiment, the system is capable of carrying out as many as 10 runs per day or more. A further object of the invention is to provide assays to survey methylation status of a genomic sequence, NBL2.

5.3 Nucleic Acids

The present invention also provides isolated polynucleotides, referred to as “CpG diagnostic polynucleotides” which are useful for characterizing tissue samples obtained from a subject suspected of having cancer. In preferred embodiments, the cancer is Wilms tumor, ovarian carcinomas, ovarian cystadenoma, neuroblastoma, hepatocellular carcinoma, or kidney cancer. The CpG diagnostic polynucleotides comprise a sequence which contains CpG dinucleotides at position(s) within the subregion of NBL2 that may either be differentially methylated or unmethylated depending on whether it is in a disease state or a normal state. In specific embodiments, the CpG diagnostic polynucleotides are 15-20 nucleic acids, 20-25 nucleic acids, 25-30 nucleic acids, 30-35 nucleic acids, 35-40 nucleic acids, 40-45 nucleic acids, 45-50 nucleic acids, 50-55 nucleic acids, 55-60 nucleic acids, 60-65 nucleic acids, 65-70 nucleic acids, 70-75 nucleic acids, 75-80 nucleic acids, 80-100 nucleic acids, 100-150 nucleic acids, 150-200 nucleic acids, 200-300 nucleic acids, 300-400 nucleic acids, 400-500 nucleic acids, 500-600 nucleic acids, 600-700 nucleic acids, 700-800 nucleic acids, 800-900 nucleic acids, or 900-1,000 nucleic acids in length. In specific embodiments, the CpG diagnostic polynucleotides are 50-60%, 60-70%, 70-80%, 80-90%, 90-100% identical to SEQ ID NO:1, 2, or 8. In other specific embodiments, the CpG diagnostic polynucleotides hybridize to a nucleic acid molecule having a nucleotide sequence of SEQ ID NO:1, 2, or 8 under stringent conditions. In specific embodiments, the CpG diagnostic polynucleotides comprises one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more CpG dinucleotides. In a specific embodiment, the CpG diagnostic polynucleotide is single-stranded. In another specific embodiment, the CpG diagnostic polynucleotide is double-stranded.

5.4 Patient Population

The invention provides methods for diagnosis or prognosis associated with cancer in a subject. The subject is preferably a mammal such as a non-primate (e.g., cattle, swine, sheep, horses, cats, dogs, rodents, etc.) and a primate (e.g., monkey and a human). In a preferred embodiment, the subject is a human. In specific embodiments, the subject is an infant, a child, or an adult.

The methods of the invention may be used to diagnose or provide prognoses to patients suffering from or expected to suffer from a hyperproliferative cell disorder, e.g., have a genetic predisposition for a hyperproliferative cell disorder or have suffered from a hyperproliferative cell disorder in the past or have been exposed to carcinogen or have been infected or previously exposed to cancer antigens. In a preferred embodiment, the patient is predisposed or is suffering from ovarian carcinoma, ovarian cystadenoma, Wilms tumor, neuroblastoma, hepatocellular carcinoma, or kidney cancer.

Such patients may or may not have been previously treated for cancer. The methods of the invention may be used as a first line or second line diagnosis or prognosis. Included in the invention is also the diagnosis or prognosis of patients currently undergoing therapies to treat cancer.

5.5 Source of a Sample

Unless otherwise indicated herein, any tissue sample (e.g., ovary or kidney) or cell sample (e.g., ovary, or kidney cell sample) obtained from any subject may be used in accordance with the methods of the invention. Examples of subjects from which such a sample may be obtained and utilized in accordance with the methods of the invention include, but are not limited to, asymptomatic subjects, subjects manifesting or exhibiting one or more symptoms of cancer, subjects clinically diagnosed as having cancer, subjects predisposed to cancer (e.g., subjects with a family history of cancer, subjects with a genetic predisposition to cancer, subjects with exposures to carcinogens, and subjects that lead a lifestyle that predisposes them to cancer or increases the likelihood of contracting cancer), subjects suspected of having cancer, subjects undergoing therapy for cancer, subjects with cancer and at least one other disease conditions, subjects not undergoing therapy for cancer, subjects determined by a medical practitioner (e.g., a physician) to be healthy or cancer-free (i.e., normal), subjects that have been cured of cancer, subjects that are managing their cancer, and subjects that have not been diagnosed with cancer. In a specific embodiment, the subjects from which a sample may be obtained and utilized have ovarian carcinoma or Wilms tumor. In another embodiment, the subjects from which a sample may be obtained and utilized have benign, malignant or metastatic cancer. A tissue biopsy by methods well-known to those skilled in the art may be obtained from a subject.

In certain embodiments, the sample obtained from a subject is from cells, cell lines, histological slides, biopsies, paraffin-embedded tissue, bodily secretions, bodily fluids, urine, cheek cell swabs, stool, blood, serum, plasma, sputum, cerebrospinal fluid, and combinations thereof. In a specific embodiment, the sample is a blood sample. A sample of blood may be obtained from a subject having any of the following developmental or disease stages of cancer. In some embodiments, a drop of blood is collected from a simple pin prick made in the skin of a subject. In such embodiments, this drop of blood collected from a pin prick is all that is needed. Blood may be drawn from a subject from any part of the body (e.g., a finger, a hand, a wrist, an arm, a leg, a foot, an ankle, a stomach, and a neck) using techniques known to one of skill in the art, in particular methods of phlebotomy known in the art. In a specific embodiment, venous blood is obtained from a subject and utilized in accordance with the methods of the invention. In another embodiment, arterial blood is obtained and utilized in accordance with the methods of the invention. The composition of venous blood varies according to the metabolic needs of the area of the body it is servicing. In contrast, the composition of arterial blood is consistent throughout the body. For routine blood tests, venous blood is generally used.

Venous blood can be obtained from the basilic vein, cephalic vein, or median vein. Arterial blood can be obtained from the radial artery, brachial artery or femoral artery. A vacuum tube, a syringe or a butterfly may be used to draw the blood. Typically, the puncture site is cleaned, a tourniquet is applied approximately 3-4 inches above the puncture site, a needle is inserted at about a 15-45 degree angle, and if using a vacuum tube, the tube is pushed into the needle holder as soon as the needle penetrates the wall of the vein. When finished collecting the blood, the needle is removed and pressure is maintained on the puncture site. Usually, heparin or another type of anticoagulant is in the tube or vial that the blood is collected in so that the blood does not clot. When collecting arterial blood, anesthetics can be administered prior to collection.

The collected sample is optionally stored at refrigerated temperatures, such 4° C., prior to use in accordance with the methods of the invention. In some embodiments, a portion of the sample is used in accordance with the invention at a first instance of time whereas one or more remaining portions of the sample is stored for a period of time for later use. This period of time can be an hour or more, a day or more, a week or more, a month or more, a year or more, or indefinitely. For long term storage, storage methods well known in the art, such as storage at cryo temperatures (e.g. below −60° C.) can be used. In some embodiments, in addition to storage of the sample, isolated nucleic acid or protein are stored for a period of time for later use. Storage of such molecules can be for an hour or more, a day or more, a week or more, a month or more, a year or more, or indefinitely.

Cells from a tissue sample or blood sample are separated from whole tissue or whole blood are collected from a subject using techniques known in the art.

Cells from a subject can be sorted using a using a fluorescence activated cell sorter (FACS). Fluorescence activated cell sorting (FACS) is a known method for separating particles, including cells, based on the fluorescent properties of the particles. See, for example, Kamarch, (1987). Methods Enzymol 151, 150-165. Laser excitation of fluorescent moieties in the individual particles results in a small electrical charge allowing electromagnetic separation of positive and negative particles from a mixture. An antibody or ligand used to detect a cell antigenic determinant present on the cell surface of particular cells is labeled with a fluorochrome, such as FITC or phycoerythrin. The cells are incubated with the fluorescently labeled antibody or ligand for a time period sufficient to allow the labeled antibody or ligand to bind to cells. The cells are processed through the cell sorter, allowing separation of the cells of interest from other cells. FACS sorted particles can be directly deposited into individual wells of microtiter plates to facilitate separation.

Magnetic beads can be also used to separate cells. For example, cells can be sorted using a using a magnetic activated cell sorting (MACS) technique, a method for separating particles based on their ability to bind magnetic beads (0.5-100 m diameter). A variety of useful modifications can be performed on the magnetic microspheres, including covalent addition of an antibody which specifically recognizes a cell-solid phase surface molecule or hapten. A magnetic field is then applied, to physically manipulate the selected beads. In a specific embodiment, antibodies to a cell surface marker are coupled to magnetic beads. The beads are then mixed with the cell culture to allow binding. Cells are then passed through a magnetic field to separate out cells having the cell surface markers of interest. These cells can then be isolated.

5.6 Cancers

Cancers and related disorders that can be diagnosed in accordance with the invention include, but are not limited to cancers of epithelial origin, endothelial origin, etc. Non-limiting examples of such cancers include the following: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, ovarian cystadenoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to pappillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypemephroma, fibrosarcoma, transitional cell cancer (renal pelvis and/or uterer); Wilms' tumor, kidney cancer; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman, et al. (1985). Medicine, 2d Ed., J.B. Lippincott Co., Philadelphia and Murphy, et al. (1997). Informed Decisions: The Complete Book of Cancer Diagnosis, Treatment, and Recovery, Viking Penguin, Penguin Books U.S.A., Inc., United States of America).

Other cancers also include breast, colon, pancreas, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Burkitt's lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoactanthoma, seminoma, thyroid follicular cancer and teratocarcinoma. Cancers caused by aberrations in apoptosis may include, but not be limited, to follicular lymphomas, carcinomas with p53 mutations, hormone dependent tumors of the breast, prostate and ovary, and precancerous lesions such as familial adenomatous polyposis, and myelodysplastic syndromes. In preferred embodiments, cancers that may be diagnosed include ovarian carcinomas, ovarian cystadenoma, Wilms tumor, kidney cancer, neuroblastoma, and hepatocellular carcinoma.

5.7 Therapeutic Agents Useful for Treatment of Cancer

In some embodiments, the invention provides methods for diagnosis and prognosis of cancer before, during and after the course of treatment of cancer in a patient. Examples of such other therapies include, but are not limited to, chemotherapy, radiation therapy, hormonal therapy and/or biological therapy and/or immunotherapy, bone marrow transplantation, and/or gene therapy.

One of the treatment for cancer is chemotherapy. The treatment includes administration of chemotherapies including, but not limited to thalidomide (THALOMID®), dexamethasone, arsenic trioxide (TRISENOX®), pamidronate, bortezomibi (VELCADE®), methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, docetaxel, carmustine, melphalan, cyclophosphamide, lenalidomide (REVLIMID™), etc. Among these patients are patients treated with radiation therapy, hormonal therapy and/or biological therapy/immunotherapy.

5.8 Kits

The invention provides kits that are useful in diagnosis and prognosis of cancer in a subject. The kits of the present invention comprise one or more probes, linkers and/or primers useful for determination of methylation status of one or more CpG dinucleotide sequences in a subregion of NBL2. The probes of the marker nucleotide sequence may be part of an array, or the probes may be packaged separately and/or individually. The kits of the present invention may also include reagents such as buffers, or other reagents that can be used in determining the methylation status of one or more CpG dinucleotide sequences in a subregion of NBL2.

In one embodiment, the invention provides kits comprising probes that are immobilized at an addressable position on a substrate, e.g., in a microarray, optionally in a sealed container.

Included in a kit of the present invention are bisulfite conversion reagents that may include: DNA denaturation buffer, sulfonation buffer, DNA recovery reagents or kits (e.g., precipitation, ultrafiltration, affinity column), desulfonation buffer, and DNA recovery components.

### 6. EXAMPLES

The present invention is further illustrated by the following examples. These examples are provided to aid in the understanding of the invention and are not construed as a limitation thereof.

### Example 1

With IRB approval, primary tumor samples were obtained from surgery patients prior to chemotherapy or radiation therapy. Informed consent was given by all patients or unlinked samples were used. The LCLs were previously described (Ehrlich, et al. (2001). Hum. Mol. Genet., 10, 2917-2931; Gisselsson, et al. (2005). Chromosoma, 114, 118-126; Tuck-Muller, et al. (2000). Cytogenet. Cell Genet., 89, 121-128; GM17900, AG14836, AG14953, and AG15022 from the Coriell Institute). Control somatic tissues were autopsy specimens of trauma victims (individuals A, B, and C, all males of 56, 19, and 68 y, respectively). DNA was purified as previously described (Ehrlich, et al. (2002). Oncogene, 21, 6694-6702).

### Example 2

Genomic methylation data were analyzed using R version 2.0.1. Chi-square test statistics were used to assess differences of proportions, and strengths of association for continuous and ordinal variables were evaluated using the standard Pearson's correlation and Kendall's tau statistics, respectively. Where appropriate, p-values were adjusted for multiple tests using the Holm procedure. Classification trees were generated using the RPART library (Breiman, et al. (1984). Classication and Regression Trees. Wadsworth: Belmont, Calif.).

### Example 3

NBL2 has a consensus sequence as set forth in SEQ ID NO:2. There are 20 copies of the NBL2 sequence in the GenBank sequence AC018692, BAC clone. The most representative of all the 20 sequences is a repeat that can be amplified using hairpin bisulfite PCR method. For example, BsmAI site is in the hairpin linker and no other BsmAI site is within subregion 1. The average number of restriction enzyme sites in the 20 copies of NBL2 sequences is as follows: AvaI: 3(2.7); BstUI:5(5.5); HhaI: 5(5.4); HpaII:9(9.1); HpyCH4IV:2(3.1); NotI: 1(0.75), the number in the bracket is the average number for the entire 20 copies of the repeat.

This Example demonstrates the contribution of spreading of methylation or demethylation to the cancer-linked methylation patterns.

Spreading of de novo methylation along a DNA region can accompany oncogenic transformation, transfection, or viral infection (Toth, et al. (1989). Proc. Natl. Acad. Sci. USA, 86, 3728-3732; Nguyen, et al. (2001). J. Natl. Cancer Inst., 93, 1465-1472; Turker, (2002). Oncogene, 21, 5388-5393; Yan, et al. (2003). Cancer Res., 63, 6178-6186). Overall, there are no evidence of predominant spreading of de novo methylation or demethylation because a pairwise comparison of neighboring CpG sites in NBL2 in the cancers indicated that there was no statistically significant bias towards adjacent sites having the same methylation status (M/M or U/U). Furthermore, at CpG6 and CpG8, which are separated by only 6 bp, there were seven clones from four cancers (OvCaN and WT4, 9, 21, and 67) that exhibited opposite methylation changes, namely increased methylation at CpG6 (M/M) and decreased methylation at CpG8 (M/U; FIGS. 3 and 4). The methylation changes in many of the clones suggest multiple discontinuous hits of demethylation and de novo methylation within a 0.2-kb region during carcinogenesis.

Nonetheless, there were some DNA clones that could be explained by spreading of methylation or demethylation in some of the NBL2 repeats. Some of these clones had all 14 CpG dyads unmethylated or all methylated (FIGS. 3 and 4). Other clones whose methylation pattern suggested spreading of demethylation had the first 5 or 6 CpG sites unmethylated on at least one strand. The frequency of clones with no methylation in the first five CpG sites was significantly higher than expected if the methylation at each site was independent, as was the combined frequency of fully methylated or fully unmethylated clones (both p-values were less than 0.0001). In summary, there seems to be spreading of altered DNA methylation patterns in some, but not most, of the copies of NBL2 in the examined cancers.

### Example 4

This Example illustrates the presence of hemimethylation in cancers.

In the examined NBL2 subregion in the somatic controls, 1.6% of the CpG sites and 15% of the somatic control clones displayed hemimethylation. The hemimethylation frequencies rose in the cancers to 3.4% of the CpG sites in 47% of the ovarian cancer clones and 6.6% of the CpG sites in 71% of the Wilms tumor clones. Because incomplete bisulfite modification was observed at only approximately 0.1% of the non-CpG C residues, these are truly hemimethylation at CpG's. There was also a change in the distribution of hemimethylation in the cancer DNAs. Hemimethylation in the examined 91 somatic control clones was seen only at 4 of the 14 CpG positions. In contrast, hemimethylation was seen at each CpG position in at least one of the 73 Wilms tumor clones and in most of the 14 CpG positions in the 73 ovarian carcinoma clones (FIGS. 3 and 4). Some of the hemimethylated CpG's at a given position displayed a strong bias for demethylation of the top or the bottom strand (Table 1). Hemimethylated CpG dyads in cancer and control clones usually did not occur as runs, but rather had the closest CpG on either side as an M/M or U/U dyad. Furthermore, of the 27 cancer clones containing more than one hemimethylated CpG site, 15 had hemimethylated dyads of opposite polarity with respect to which strand was unmethylated.

TABLE 1
Examples of asymmetry in hemimethylation
of NBL2 sites in cancers

| | No. of clones with indicated | | |
|---|---|---|---|
| | type of demethylation at: | | |
| Dyad methylation status | CpG8 | CpG10 | CpG12 |
| Symmetrically demethylated | 9 | 22 | 49 |
| (demeth. in both strands) | | | |
| Hemimethylated (demeth. | 9 | 18 | 11 |
| in only one strand) | | | |
| Hemimethylated as U/M | 1 | 18 | 9 |
| (demeth. only in top strand)^{a} | | | |
| Hemimethylated as M/U | | | |
| (demeth. only in bottom strand)^{b} | | | |
| | 8 | 0 | 11 |

^{a}U/M status at CpG10 was seen in five cancers.
^{b}M/U status at CpG8 was seen in five cancers and at CpG12 in four cancers.

### Example 5

In order to explain site preferences for cancer-linked methylation changes or for the conserved methylation patterns in somatic controls, possible effects of the sequence 1-3 bp on either side of each CpG were investigated. No rules for predicting the methylation status in the somatic controls or cancers based upon adjacent sequences could be deduced just from the region subjected to genomic sequencing. However, Southern Blot analysis of NBL2 arrays gave us further insights, as described in the following Example. For Southern blot analysis, 1.5 μg of human DNA was digested with 15-30 U of restriction endonuclease overnight according to the manufacturer's procedures (New England Biolabs), all with parallel internal controls as previously (Nishiyama, et al. (2005). Cancer Biol. Ther., 4, 440-448). At least three diverse somatic control tissues and sperm DNA were included as references in each blot.

### Example 6

This Example illustrates the analysis of CpG methylation by Southern blotting.

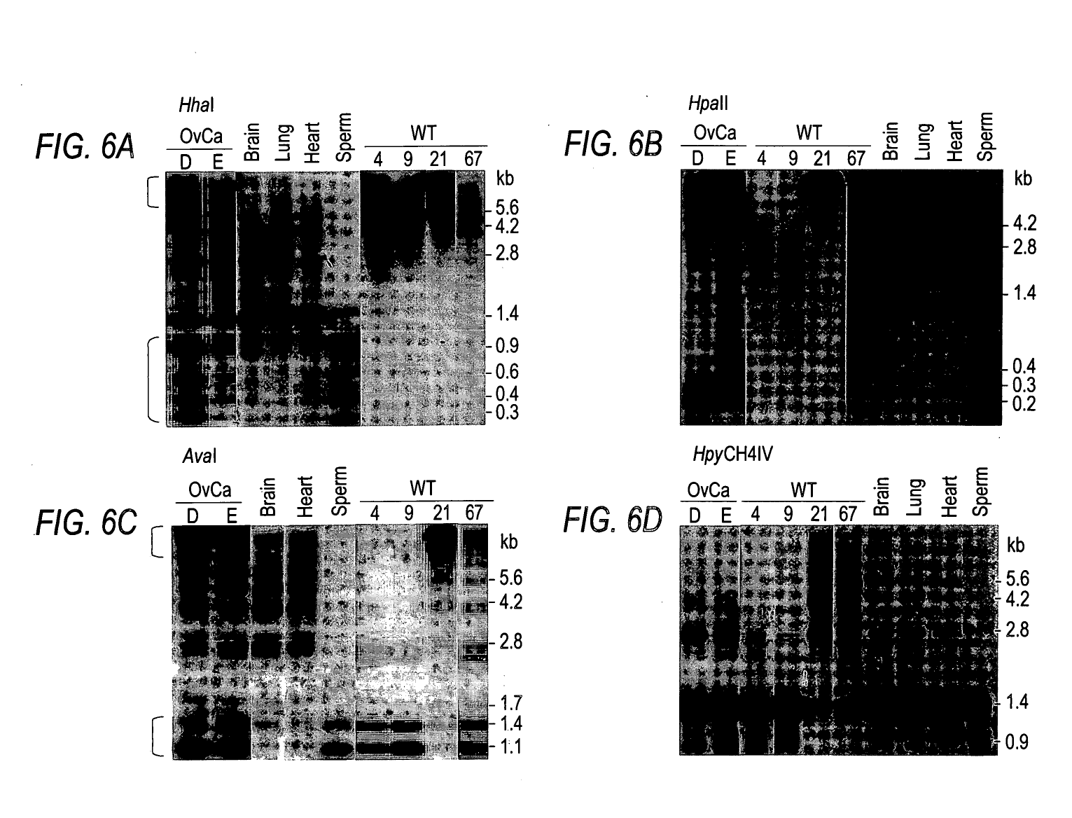

All cancers in this study and an additional 13 ovarian carcinomas and 46 Wilms tumors had been examined by Southern Blot analysis for methylation at HhaI and NotI sites with a 1.4-kb NBL2 probe (Nishiyama, et al. (2005). Cancer Biol. Ther., 4, 440-448). For Southern blot analysis, 1.5 μg of human DNA was digested with 15-30 U of restriction endonuclease overnight according to the manufacturer's procedures (New England Biolabs), all with parallel internal controls as previously (Nishiyama, et al. (2005). Cancer Biol. Ther., 4, 440-448). At least three diverse somatic control tissues and sperm DNA were included as references in each blot. HhaI digests of DNAs from various postnatal somatic control tissues from 15 individuals gave very similar distributions of intermediate-molecular-weight hybridizing fragments (e.g., see FIG. 6A), while NotI digests all gave very high-molecular-weight hybridizing fragments (Nishiyama et al., 2005; and unpub. data). A comparison of HhaI digests of cancers and somatic controls revealed predominant hypermethylation in most of the cancers and hypomethylation in others (e.g., FIG. 6A). Advantages of Southern Blot analysis are that it can show long-range methylation patterns not identifiable by genomic sequencing, especially in tandem repeats, and it provides results from the population average of all the copies of the examined sequence.

First, cancers for Southern Blot-determined methylation changes at HhaI sites throughout the NBL2 arrays and sequencing-determined methylation changes in the 0.2-kb NBL2 subregion were examined. HhaI-site methylation scores were approximated from phosphorimager quantitation of Southern Blot results (+1 to +3, increasing hypermethylation relative to somatic controls; −1 to −3, increasing hypomethylation, Table 2; Nishiyama, et al. (2005). Cancer Biol. Ther., 4, 440-448). The genomic sequencing data for each cancer clone were quantified as the weighted average of hypermethylation at the two normally unmethylated CpG's and hypomethylation at the seven normally methylated CpG's. There was a significant association between NBL2 methylation changes in the cancers determined by these two assays (p<0.001). Therefore, both are monitoring similar methylation changes. Also, a comparison of the overall 5-Methylcytosine content of the DNA (by HPLC analysis) and the total proportion of methylated sites in the NBL2 0.2-kb subregion indicated a significant association between these two epigenetic parameters (p=0.001) as well as between global DNA methylation and NBL2 HhaI-site methylation. Therefore, NBL2 methylation changes in cancer are linked to global DNA methylation changes.

In order to analyze CpG methylation in cancer DNAs at HpaII, AvaI, HpyCH4IV, or BstUI sites (FIG. 2), methylation of normal somatic DNAs were analyzed. DNAs were digested with these CpG methylation-sensitive enzymes and probed with the 1.4-kb NBL2 sequence. All somatic controls from various postnatal somatic tissues derived from different individuals gave very similar Southern Blot results with a given enzyme. However, there was much more resistance of NBL2 arrays in all these controls to cleavage by some of these enzymes than by others (FIG. 6). These results could not be explained by the frequency of the restriction sites in NBL2. For example, there are an average of about 9-10 HpaII sites vs. 5-6 HhaI, 2-4 AvaI, 3-5 HpyCH4IV, and 6 BstUI sites per NBL2 monomer (GenBank Y10752 and AC0128692). Nonetheless, NBL2 arrays in somatic controls were much more resistant to digestion by HpaII than by the other enzymes, and HpyCH4IV gave more cleavage than the others enzymes (FIG. 6). The low extent of digestion of NBL2 arrays by HpaII in somatic control DNAs was not due to sequence variation by showing complete digestion of all tested samples to <0.4-kb fragments by MspI, an isoschizomer of HpaII. MspI is resistant to CpG methylation except at GGCCGG sites (Busslinger et al., 1983). Also, internal controls for the HpaII digests, which were used for all digests, showed that no inhibitors were present. The preferential methylation of NBL2 HpaII sites in somatic controls observed in Southern Blot assays was consistent with genomic sequencing data (FIG. 3, CpG2, CpG5, and CpG11).

All 18 ovarian cancer DNAs and 13 of the 15 Wilms tumor DNAs examined with at least 3 of the above enzymes exhibited altered Southern Blot patterns of NBL2 methylation relative to somatic controls (Table 2). Southern Blot data from cancer DNAs digested with different enzymes were shown (FIG. 6) with the caveat that HpaII digests give an underestimate of hypermethylation, and HpyCH4IV digests give an underestimate of hypomethylation. Importantly, HhaI sites appeared to undergo de novo methylation during carcinogenesis more frequently than AvaI, HpyCH4IV, and BstUI sites despite all of these enzymes giving mostly intermediate-molecular-weight NBL2-hybridizing bands in somatic controls (FIG. 6, Table 2). This suggests some sequence specificity to cancer-linked hypermethylation. In addition, the distribution of NBL2-containing restriction fragments in HhaI digests and AvaI digests of ovarian carcinomas D and E indicated that NBL2 arrays can be bifurcated into two epigenetic components differing in the extent of methylation at a given restriction site (brackets in FIGS. 6A and C). Long tandem regions of hypermethylation at these two kinds of restriction sites were observed as increases in NBL2 signal in >10-kb fragments even though those tumors also displayed increases in low-molecular weight signal relative to the somatic controls. Separate fractions of NBL2 repeats with respect to long-range methylation patterns might correspond to NBL2 arrays on different acrocentric chromosomes.

TABLE 2
Methylation changes in NBL2 repeats in Wilms tumors and ovarian carcinomas relative
to somatic controls as determined by hairpin sequencing or Southern blot analysis

| | Summary of genomic | | | | | | Global DNA |
|---|---|---|---|---|---|---|---|
| | sequencing results ^{a} | | NBL2 methylation scores from SB with | | | | methylation ^{c} |
| | Hypermeth. | Hypometh. | the indicated DNA digests ^{b} | | | | % C |
| Sample | (%) | (%) | Hha I | Ava I | Hpa II | Bst UI | methylated |
| | | | | [symbol] [symbol] | [symbol] | [symbol] [symbol] | |
| Ovarian care. D | 22 | 50 | −2 | ↓ | ↓ | ↓ | 3.31 |
| Ovarian care. E | 33 | 47 | −1 | ↓ | ↓ | ↓ | 2.94 |
| Wilms tumor 9 | 33 | 26 | +1 | ↓ | ↓ | ↓ | 3.09 |
| Wilms tumor 4 | 50 | 30 | +1 | ↓ | ↓ | NC | 2.88 |
| Ovarian carc. N | 63 | 11 | +2 | NC | ↓ | ↑ | 3.76 |
| Wilms tumor 67 | 78 | 9.3 | +1 | ↓ | ↓ | ↓ | 3.45 |
| Ovarian carc. O | 81 | 9.3 | +2 | ↓ | ↓ | ↑ | 3.73 |
| Wilms tumor 21 | 86 | 4.9 | +3 | ↑ | ↑ | ↑ | 3.90 |
| Ovarian carc. Q | 87 | 14 | +3 | NC | ↓ | ↑ | 3.57 |
| Wilms tumor 16 | 89 | 5.3 | +3 | ↑ | ↑ | ↑ | 3.67 |
| ICF B LCL | 19 | 53 | −3 | ↓ | ↓ | ND | ND |
| Pat C LCL | 63 | 12 | +2 | ↓ | NC | ND | ND |
| ^{a} Hypermethylation in NBL2 at the normally unmethylated CpG6 and CpG14 in the cancers and in an | | | | | | | |
| # ICF LCL (ICF B) and a control (Pat C) was calculated as the overall percentage of these two sites with | | | | | | | |
| # symmetrical methylation. Hypomethylation was calculated as the overall loss of symmetrical methylation | | | | | | | |
| # at the normally methylated CpG sites 2, 3, 5, 8, 10, 11, and 12. | | | | | | | |
| ^{b} Methylation scores from Hha I digests of cancer and LCL DNAs relative to somatic controls were | | | | | | | |
| # from previous SB analyses with phosphorimager quantitation (Nishiyama et al., 2005). Negative values | | | | | | | |
| # denote overall hypomethylation at HHa I sites and positive values, overall hypermethylation at these sites. | | | | | | | |
| # For the other CpG methylation-sensitive enzymes, downward and upward arrows denote hypomethylation and | | | | | | | |
| # hypermethylation, respectively. NC, no change in methylation relative to the somatic controls; ND, not | | | | | | | |
| # determined. | | | | | | | |
| ^{c} Global genomic methylation levels determined by HPLC analysis of DNA digested to mononucleosides | | | | | | | |
| # (Ehrlich et al., 2002; and unpub. data). Depending on the tissue, somatic controls have 3.43-4.04% of | | | | | | | |
| # genomic C residues methylated. | | | | | | | |

### Example 7

This Example shows the involvement of DNMT3B in methylation of NBL2.

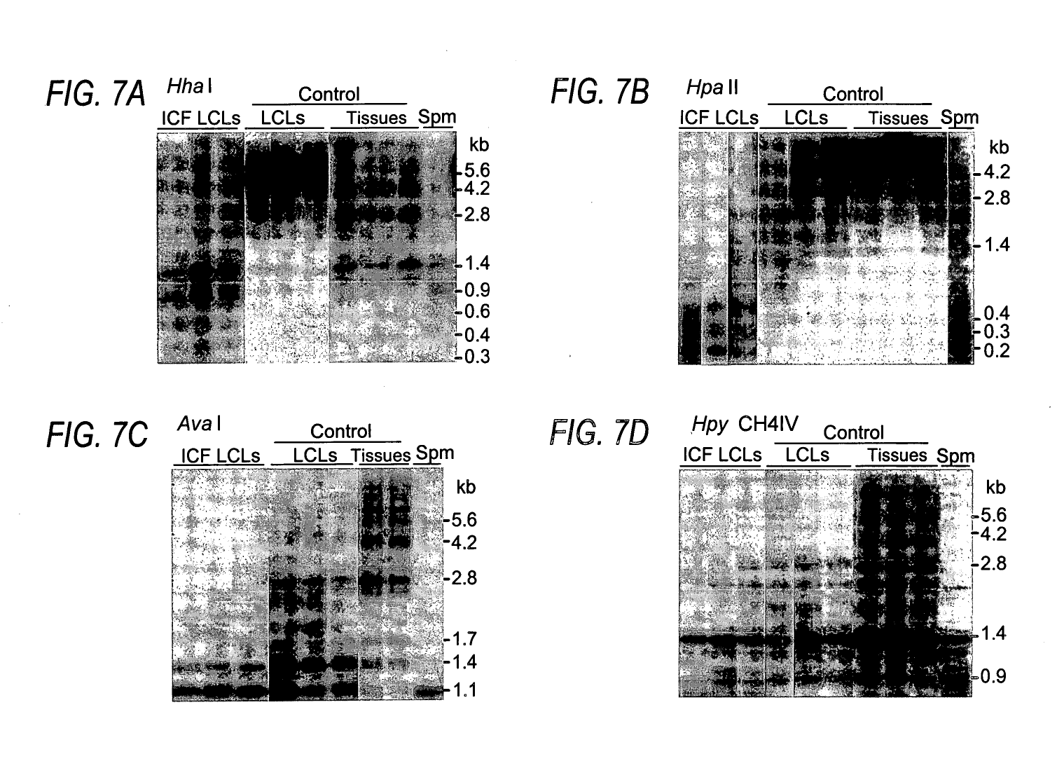

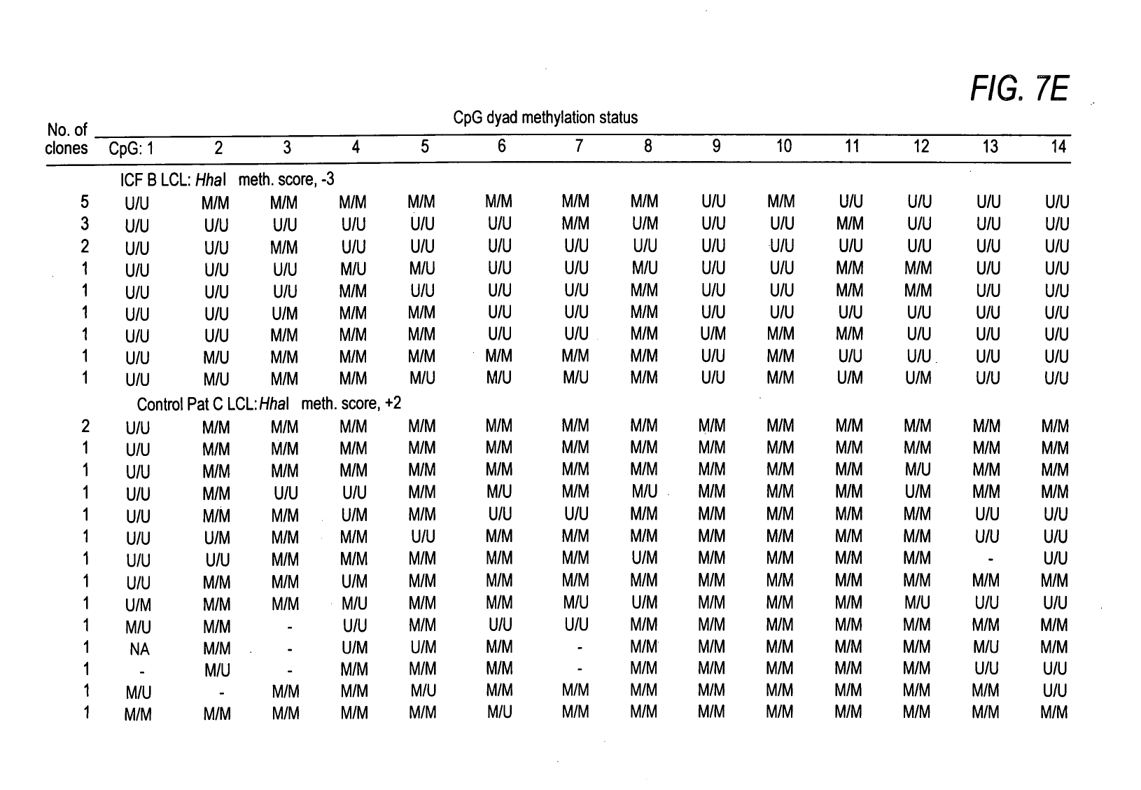

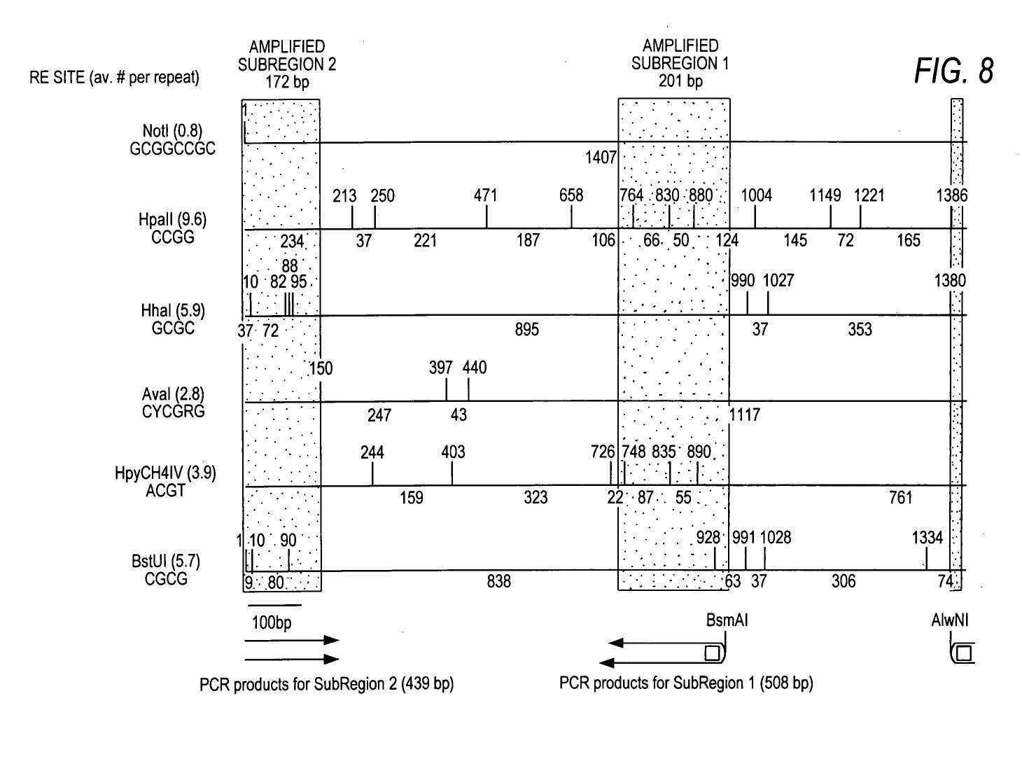

ICF syndrome patients usually have missense DNMT3B mutations in both alleles (Hansen, et al. (1999). Proc. Natl. Acad. Sci. USA, 96, 14412-14417; Okano, et al. (1999). Cell, 98, 247-257; Xu, et al. (1999). Nature, 402, 187-191), which greatly reduce enzymatic activity (Gowher, et al. (2002). J. Biol. Chem., 277, 20409-20414). To examine the involvement of DNMT3B in methylation of NBL2, Southern Blot analysis of DNA digests were performed from six ICF B-cell lines, known to have DNMT3B mutations, and ten control B-cell lines. Relative to normal somatic tissues, hypomethylation at NBL2 HhaI sites was seen in four of the six ICF lymphoblastoid cell lines (LCLs) but none of the ten control LCLs. Instead, the control LCLs were hypermethylated in NBL2 arrays compared to normal somatic tissues, including leukocytes (FIG. 7A). This indicates that NBL2 underwent de novo methylation at HhaI sites during generation or passage of LCLs only if the LCLs had normal DNMT3B activity.

All but one of the ICF LCLs displayed hypomethylation at NotI sites in NBL2 arrays while none of the control LCLs did. In addition, the ICF LCLs showed hypomethylation at HpaII sites compared with control LCLs and control somatic tissues (FIG. 7B). However, both control and ICF LCLs exhibited hypomethylation at AvaI and HpyCH4IV sites compared to control somatic tissues (FIGS. 7C and 7D). Relative to normal somatic tissues, there was hypomethylation and hypermethylation at individual CpG's in NBL2 in LCLs from ICF patients B and C and a control LCL by genomic sequencing (FIG. 7E), although there was more hypomethylation in the ICF cells and more hypermethylation in the control cells (Table 2).

### Example 8

This Example investigates the transcription of NBL2.

Eight diverse somatic tissues and 16 out of 20 cancers (ovarian carcinomas and Wilms tumors) does not express NBL2 as determined by RT-PCR (Nishiyama, et al. (2005). Cancer Biol. Ther., 4, 440-448). Using random primers in one set and oligo(dT) in a duplicate set, cDNA was synthesized from 3 μg of total RNA that had been treated with 3 U of DNase I (Amplification Grade, Invitrogen) for 45 min at room temperature. Real-time PCR (SYBR green PCR Master Mix, Applied Biosystems) was done with previously described primers and conditions (Nishiyama, et al. (2005). Cancer Biol. Ther., 4, 440-448). Semi-quantitative RT-PCR with evaluation of the product by gel electrophoresis was also done as previously described (Nishiyama, et al. (2005). Cancer Biol. Ther., 4, 440-448). The four positive cancers and one tested LCL (ICF B) evidenced transcripts by both real-time and semi-quantitative RT-PCR, but only at low levels. There was no relationship to hypomethylation at HhaI sites in NBL2, and NBL2 RNA was shown to probably result from run-through transcription. Five ICF LCLs and ten control LCLs were tested for NBL2 transcripts by real-time RT-PCR with GAPDH transcripts as the internal standard. Low levels of NBL2 RNA were seen in the four ICF LCLs that displayed hypomethylation at HhaI sites. Neither of the other two ICF LCLs and none of the control LCLs gave a signal appreciably above background, and also none of these displayed hypomethylation at HhaI sites. Duplicate cDNAs prepared from each LCL with random primers or oligo(dT) gave similar results in real-time RT-PCR. Also, semi-quantitative RT-PCR confirmed that the correct size product was obtained from an ICF LCL (ICF C) using either oligo(dT) or random priming, and no product was obtained from a control LCL (Pat C). Product formation from the ICF LCL was shown to be dependent on reverse transcription. An unspecified promoter adjacent to one of the NBL2 arrays might be hypomethylated in the NBL2 RNA-positive ICF LCLs and cancers and thereby activated for run-through transcription.

The tandem 1.4-kb NBL2 repeat provided new insights into several aspects of epigenetics in normal tissues and cancers. Itano, et al. (2002). Oncogene, 21, 789-797. In the 0.2-kb subregion of NBL2 from diverse control somatic tissues that was examined by hairpin-bisulfite genomic sequencing, there was a completely conserved pattern of undermethylation at two non-adjacent CpG's and full methylation at seven other CpG's (FIG. 5A). This methylation pattern was lost in all 146 DNA clones from ten cancers (ovarian carcinomas and Wilms tumors). Moreover, all but two of the cancer clones were hypomethylated at one specific CpG dyad or hypermethylated at another dyad only 14 bp away (CpG6 and 10). None of the normal DNA clones had this epigenetic signature. The two exceptional cancer clones lacked one of these CpG's because of sequence variation, but the methylation status of a third CpG (CpG14) allowed those two clones to be distinguished from all normal clones. Hypermethylation at CpG6 and/or CpG14 as well as hypomethylation at CpG2, 3, 5, 8, 10, 11, and/or 12 were seen in the majority of cancer DNA clones. Although hypermethylation of CpG6 or hypomethylation of CpG10 were the most diagnostic epigenetic changes for cancer, there was only one cancer DNA clone (in WT67, FIG. 4) that displayed both hypermethylation and hypomethylation at precisely these positions. This observation and the non-random nature of many of the other DNA methylation changes observed by genomic sequencing and SB analysis indicate that losses and gains of methylation in NBL2 during carcinogenesis are often targeted to specific CpG positions and in specific patterns within the repeat.

The targeting of NBL2 for non-random hypermethylation and hypomethylation cannot be explained by transcription-related binding of sequence-specific DNA binding proteins, as is the case for certain promoters (Hornstra, et al. (1994). Mol. Cell. Biol., 14, 1419-1430). NBL2 underwent extensive cancer-linked alterations in methylation despite its lack of transcription in normal tissues and in most analyzed cancers and absence of an in silico-predicted gene structure (Nishiyama, et al. (2005). Cancer Biol. Ther., 4, 440-448). Therefore, silencing of transcription is not necessary for all cancer-associated DNA hypermethylation although it has been implicated in promoter hypermethylation (Clark, et al. (2002). Oncogene, 21, 5380-5387). Moreover, an in silico search for consensus sites for sequence-specific DNA-binding proteins in NBL2 (TESS: Transcription Element Search Software) did not yield putative sites that could explain the observed methylation patterns.

Cancer-linked demethylation of NBL2 was often observed in more than one of the seven normally methylated CpG positions with intervening CpG's that retained methylation. With respect to hemimethylation, cancer clones had a higher frequency of hemimethylated CpG sites than somatic control clones, and these included clones with two hemimethylated sites having opposite strands unmethylated. These results indicate that demethylation by inhibition of maintenance methylation after DNA replication is not the major source of cancer-linked hypomethylation. Instead they suggest some kind of active demethylation. The mechanism for demethylation in cancer is uncertain; however, it is clear that mammals have the capacity for active demethylation, as seen in the male pronucleus of the mouse zygote (Santos, et al. (2002). Dev. Biol., 241, 172-182).

With regard to de novo methylation of NBL2 in cancer, DNMT3B is likely to be the main enzyme involved, as determined by our analysis of B-cell LCLs from controls and from ICF patients. ICF patients usually have inactivating mutations in DNMT3B that eliminate most DNMT3B activity (Gowher, et al. (2002). J. Biol. Chem., 277, 20409-20414). The much lower levels of NBL2 methylation in ICF LCLs than in control LCLs implicate DNMT3B in establishing the normal NBL2 methylation pattern during development. The hypermethylation of NBL2 at HhaI sites in control LCLs relative to somatic control tissues could be explained by overexpression of DNMT3B (as well as DNMT3A and DNMT1) during transformation with Epstein-Barr virus (Tsai, et al. (2002). Proc. Natl. Acad. Sci. U.S.A., 99, 10084-10089). In vitro transformation of lymphocytes by Epstein-Barr virus may provide a good model for understanding NBL2 methylation changes during malignant transformation in vivo because both hypomethylation and hypermethylation relative to control somatic tissues was observed in NBL2 in normal LCLs. In the two ICF LCLs subject to genomic sequencing, despite the overall hypomethylation of NBL2, some hypermethylation was observed at CpG6, although not at CpG14, the other site at which we could analyze cancer-linked hypermethylation (FIG. 7E). Also, the control LCL displayed more methylation at CpG6 than CpG14. Similarly, CpG6 was hypermethylated significantly more frequently than CpG14 in ovarian carcinomas. Moreover, CpG6 was occasionally hemimethylated in somatic controls while CpG14 was always symmetrically unmethylated. These findings might be related to the dynamic system of normal maintenance methylation and de novo methylation proposed by Pfeifer et al. (Pfeifer, et al. (1990). Proc. Natl. Acad. Sci. USA, 87, 8252-8256). At NBL2, there may be infrequent de novo methylation of CpG6 in one strand in normal cells, which is not followed by maintenance methylation. In contrast, there may be frequent hemimethylation at this site with subsequent maintenance methylation upon oncogenic transformation.

An in vitro study of methylation by Dnmt3b indicated strong sequence preferences for de novo methylation (Handa, et al. (2005). J. Mol. Biol., 348, 1103-1112). DNMT3B/Dnmt3b may have its sequence preference strongly altered in vivo. Both the genomic sequencing and Southern Blot analysis indicate that HpaII sites (CCGG) have an especially high level of methylation in NBL2 in normal somatic tissues. Also, the Southern Blot analysis suggests that HhaI sites (which were missing from the bisulfite-sequenced region) were more frequently de novo methylated in the cancers than HpyCH4IV (AGCT), AvaI (CYCGRG), and BstUI (CGCG) sites.

### 7. EQUIVALENTS

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

1. A method for detecting or diagnosing cancer in a subject, the method comprising:
(a) determining the methylation status at one or more CpG dinucleotides of NBL2 of each strand of a double stranded genomic nucleic acid molecule in a biological sample obtained from said subject at one or more CpG dinucleotide sequences of the NBL2, and (b) comparing the methylation status of each strand of the double stranded genomic nucleic acid molecule at one or more CpG dinucleotide sequences of the NBL2 in the sample to the methylation status of each strand of a double stranded genomic nucleic acid molecule from a reference sample at the corresponding one or more genomic CpG dinucleotide sequences, wherein a difference in the methylation status of each strand of the double stranded genomic nucleic acid molecule at one or more CpG dinucleotide sequences in the sample compared to the reference indicates a change in methylation status.

2. The method of claim 1 wherein the NBL2 sequence has a nucleotide sequence of SEQ ID NO:2 or a nucleotide sequence that is at least 80% identical to SEQ ID NO:2.

3. The method of claim 1 wherein the NBL2 comprises a subregion having a nucleotide sequence of SEQ ID NO:1 or 8.

4. The method of claim 1 wherein the one or more CpG dinucleotide sequences are CpG2, CpG3, CpG5, CpG6, CpG8, CpG10, CpG11, CpG12, CpG13, or CpG14.

5. The method of claim 4 wherein the one or more CpG dinucleotide sequences are CpG6, CpG10, or CpG14.

6. The method of claim 4 wherein the methylation status of one or more genomic CpG dinucleotide sequences from CpG2, CpG3, CpG5, CpG8, CpG10, CpG11, or CpG12 in the reference are symmetrically methylated.

7. The method of claim 4 wherein the methylation status of the genomic CpG dinucleotide sequence from CpG6 in the reference is symmetrically unmethylated or asymmetrically methylated.

8. The method of claim 4 wherein the methylation status of the genomic CpG dinucleotide sequence from CpG10 in the reference is symmetrically methylated.

9. The method of claim 4 wherein the methylation status of the genomic CpG dinucleotide sequence from CpG14 in the reference is symmetrically unmethylated.

10. The method of claim 4 wherein the methylation status of the genomic CpG dinucleotide sequence from CpG13 in the reference is symmetrically methylated.

11. The method of claim 4 wherein one or more asymmetrically methylated or symmetrically unmethylated genomic CpG dinucleotide sequences from CpG2, CpG3, CpG5, CpG8, CpG10, CpG11, or CpG12 in the sample indicate a change in methylation status.

12. The method of claim 4 wherein a symmetrically methylated CpG6 in the sample indicates a variation in methylation status.

13. The method of claim 4 wherein an asymmetrically methylated or symmetrically unmethylated CpG10 in the sample indicates a change in methylation status.

14. The method of claim 4 wherein a symmetrically methylated CpG14 in the sample indicates a change in methylation status.

15. The method of claim 1 wherein when the change in methylation status is predictive of the presence or susceptibility of cancer in the subject.

16. The method of claim 15 wherein the cancer is ovarian carcinoma or Wilms tumor.

17. The method of claim 1 wherein the biological sample is from cells, cell lines, histological slides, biopsies, paraffin-embedded tissue, bodily secretions, bodily fluids, urine, cheek cell swabs, stool, blood, serum, plasma, sputum, cerebrospinal fluid, and combinations thereof.

18. The method of claim 15 wherein the predicative accuracy of cancer is greater than about 80%.

19. The method of claim 1 wherein the methylation status of one or more CpG dinucleotide sequences is determined in a method comprising the steps of: (a) treating the genomic DNA with a bisulfite reagent; (b) contacting the genomic DNA with an amplification enzyme and at least two primers that hybridizes to a nucleic acid molecule comprising a portion of the nucleotide sequence of SEQ ID NO:1 or 8, or is at least 80% identical to SEQ ID NO:1 or 8; and (c) determining the methylation status of one or more CpG dinucleotide sequence in the genomic DNA.

20. The method of claim 19 wherein said linker comprises the sequence of SEQ ID NO:3, 4, or 9.

21. The method of claim 19 wherein said primers comprise the sequence of SEQ ID NO:5, 6, 7, 10, 11, or 12.

22. The method of claim 1 further comprising a step of obtaining a biological sample comprising the genomic nucleic acid molecule from the subject.

23. A kit useful to practice the method according to claim 1.

24. A method for detecting or diagnosing cancer in a subject by identifying one or more changes in methylation status, the method comprising: (a) determining the methylation status at one or more CpG dinucleotides of NBL2 in a biological sample obtained from said subject at one or more CpG dinucleotide sequences of the NBL2, and (b) comparing the methylation status of one or more CpG dinucleotide sequences of the NBL2 in the sample to the methylation status from a reference sample at the corresponding one or more genomic CpG dinucleotide sequences, wherein a difference in the methylation status of one or more CpG dinucleotide sequences in the sample compared to the reference indicates a change in methylation status, and wherein the one or more CpG dinucleotide sequences are CpG2, CpG3, CpG5, CpG6, CpG8, CpG10, CpG11, CpG12, CpG13, CpG14, CpG21, CpG22, CpG23, CpG24, CpG25, CpG26, CpG27, CpG28, CpG29, CpG30, CpG31, CpG32, CpG33, CpG34, CpG35, CpG36, or CpG37.

25. The method of claim 24 wherein the one or more CpG dinucleotide sequences are CpG6, CpG10, or CpG14.

26. The method of claim 24 wherein the methylation status of one or more genomic CpG dinucleotide sequences from CpG2, CpG3, CpG5, CpG8, CpG10, CpG11, or CpG12 in the reference are symmetrically methylated.

27. The method of claim 24 wherein the methylation status of the genomic CpG dinucleotide sequence from CpG6 in the reference is symmetrically unmethylated or asymmetrically methylated.

28. The method of claim 24 wherein the methylation status of the genomic CpG dinucleotide sequence from CpG10 in the reference is symmetrically methylated.

29. The method of claim 24 wherein the methylation status of the genomic CpG dinucleotide sequence from CpG14 in the reference is symmetrically unmethylated.

30. The method of claim 24 wherein the methylation status of the genomic CpG dinucleotide sequence from CpG13 in the reference is symmetrically methylated.

31. The method of claim 24 wherein one or more asymmetrically methylated or symmetrically unmethylated genomic CpG dinucleotide sequences from CpG2, CpG3, CpG5, CpG8, CpG10, CpG11, or CpG12 in the sample indicate a change in methylation status.

32. The method of claim 24 wherein a symmetrically methylated CpG6 in the sample indicates a change in methylation status.

33. The method of claim 24 wherein an asymmetrically methylated or symmetrically unmethylated CpG10 in the sample indicates a change in methylation status.

34. The method of claim 24 wherein a symmetrically methylated CpG14 in the sample indicates a change in methylation status.

35. The method of claim 1 wherein when the change in methylation status is predictive of the presence or susceptibility of cancer in the subject.

36. The method of claim 35 wherein the cancer is ovarian carcinoma or Wilms tumor.

37. The method of claim 24 wherein the biological sample is from cells, cell lines, histological slides, biopsies, paraffin-embedded tissue, bodily secretions, bodily fluids, urine, cheek cell swabs, stool, blood, serum, plasma, sputum, cerebrospinal fluid, and combinations thereof.

38. The method of claim 35 wherein the change in methylation is at least −60%, −40%, −20%, 20%, 40%, 60% or 80%.

39. The method of claim 24 wherein the methylation status of one or more CpG dinucleotide sequences is determined in a method comprising the steps of: (a) treating the genomic DNA with a bisulfite reagent; (b) amplifying a portion of the NBL2 sequence; and (c) determining the methylation status of the amplified sequence by pyrosequencing.
